(19) United States
(12) Patent Application Publication
(10) Pub. No.: US 2018/0369241 A1
(43) Pub. Date: Dec. 27, 2018

(54) TREATMENT OF CANCER WITH DNAPK INHIBITORS

(71) Applicants: Celgene Corporation, Summit, NJ (US); The University of Michigan, Ann Arbor, MI (US)

(72) Inventors: Felix Yi-Chung FENG, Ann Arbor, MI (US); Ellen FILVAROFF, San Francisco, CA (US); Kristen Mae HEGE, Burlingame, CA (US); Vishal KOTHARI, Ann Arbor, MI (US); Shuang ZHAO, Ann Arbor, MI (US)

(21) Appl. No.: 15/739,159

(22) PCT Filed: Jun. 23, 2016
(86) PCT No.: PCT/US16/38888

§ 371 Date: Dec. 22, 2017

Related U.S. Application Data
(60) Provisional application No. 62/183,920, filed on Jun. 24, 2015.

(51) Int. Cl. A61K 31/4985; A61P 35/02

(52) U.S. Cl. CPC A61K 31/4985; A61K 31/4166; A61K 2300/00; A61P 35/02

(57) ABSTRACT

Provided herein are methods for treating or preventing Wnt-associated cancers, comprising administering an effective amount of a DNAPK inhibitor to a patient having a Wnt-associated cancer.

This application claims the benefit of priority to U.S. Provisional Application Ser. No. 62/183,920, filed Jun. 24, 2015, which is incorporated herein by reference in its entirety and for all purposes.

### 1. FIELD

Provided herein are methods for treating or preventing Wnt-associated cancers, comprising administering an effective amount of a DNAPK inhibitor to a patient having a Wnt-associated cancer.

### 2. BACKGROUND

Kinases play a vital role in driving oncogenic pathways and have been the mainstay in the development of therapeutics across multiple cancers (Rask-Andersen, M., et al., Advances in kinase targeting: current clinical use and clinical trials. Trends Pharmacol Sci, 2014. 35(11): p. 604-20; Zhang, J., P. L. Yang, and N. S. Gray, Targeting cancer with small molecule kinase inhibitors. Nat Rev Cancer, 2009. 9(1): p. 28-39).

DNAPK, a nuclear serine/threonine protein kinase, has been known for its role in DNA repair via the non-homologous end joining (NHEJ) pathway. However, emerging studies indicate the importance of DNAPK in a variety of other processes, including the modulation of chromatin structure and transcription through its interaction with a variety of receptors and transcription factors (Munoz, D. P., M. Kawahara, and S. M. Yannone, An autonomous chromatin/DNA-PK mechanism for localized DNA damage signaling in mammalian cells. Nucleic Acids Res, 2013. 41(5): p. 2894-906; Pyun, B. J., et al., Mutual regulation between DNA-PKcs and Snail1 leads to increased genomic instability and aggressive tumor characteristics. Cell Death Dis, 2013. 4: p. e517; Brenner, J. C., et al., Mechanistic rationale for inhibition of poly(ADP-ribose) polymerase in ETS gene fusion-positive prostate cancer. Cancer Cell, 2011. 19(5): p. 664-78; An, J., et al., Downregulation of c-myc protein by siRNA-mediated silencing of DNA-PKcs in HeLa cells. Int J Cancer, 2005. 117(4): p. 531-7; Achanta, G., et al., Interaction of p53 and DNA-PK in response to nucleoside analogues: potential role as a sensor complex for DNA damage. Cancer Res, 2001. 61(24): p. 8723-9; Bandyopadhyay, D., et al., Physical interaction between epidermal growth factor receptor and DNA-dependent protein kinase in mammalian cells. J Biol Chem, 1998. 273(3): p. 1568-73). More recently, in the context of prostate cancer, it was demonstrated that DNAPK can also transcriptionally activate the androgen receptor, potentiates AR function and thus represents a potential therapeutic target in CRPC (Goodwin, J. F., et al., A hormone-DNA repair circuit governs the response to genotoxic insult. Cancer Discov, 2013. 3(11): p. 1254-71). However, if the role of DNAPK in prostate cancer progression is just to stimulate the androgen receptor, then androgen-directed therapies should also suppress the oncogenic role of DNAPK. Given that DNAPK expression is strongly associated with metastatic CRPC progression, it is clear that DNAPK plays additional important roles in activating compensatory signaling pathways responsible for bypassing the conventional androgen-directed therapies.

The embodiments provided herein are based on the discovery of a novel role of DNAPK in regulating Wnt signaling, a mechanism which is known to play oncogenic roles across multiple cancers, including CRPC. This discovery demonstrates a need for compounds useful for treating Wnt-associated cancers.

Citation or identification of any reference in Section 2 of this application is not to be construed as an admission that the reference is prior art to the present application.

### 3. SUMMARY

Provided herein are methods for treating or preventing Wnt-associated cancers, comprising administering an effective amount of a DNAPK inhibitor to a patient having Wnt-associated cancer.

Further provided herein are methods for inhibiting or preventing metastasis of Wnt-associated cancers, comprising administering an effective amount of a DNAPK inhibitor to a patient having a Wnt-associated cancer.

Further provided herein are methods for inhibiting or preventing expansion or survival of cancer stem cells of Wnt-associated cancers, comprising contacting the cancer stem cells of a Wnt-associated cancer with an effective amount of a DNAPK inhibitor.

Further provided herein are methods for inhibiting or preventing expansion or survival of resistant and/or refractory tumor cells of Wnt-associated cancers, comprising contacting the tumor cells of the Wnt-associated cancer with an effective amount of a DNAPK inhibitor.

Further provided herein are methods for treating or preventing androgen deprivation therapy (ADT)-resistant cancers, comprising administering an effective amount of a DNAPK inhibitor to a patient having an androgen deprivation therapy-resistant cancer.

Further provided herein are methods for preventing androgen deprivation therapy resistance in cancers, comprising administering an effective amount of a DNAPK inhibitor to a patient having cancer.

Further provided herein are methods for treating or preventing enzalutamide-resistant cancers, comprising administering an effective amount of a DNAPK inhibitor to a patient having an enzalutamide-resistant cancer.

Further provided herein are methods for detecting or measuring the inhibition of DNAPK activity in a patient, comprising measuring decreased phosphorylation of a DNAPK substrate (such as DNAPK or Hsp90a) in a biological sample from said patient, for example a peripheral blood or tumor sample, prior to and after the administration of a DNAPK inhibitor to said patient.

Further provided herein are methods for detecting or measuring the effect of inhibition of DNAPK activity on markers of Wnt activity in a patient, comprising measuring markers of Wnt activity in a biological sample from said patient, for example a peripheral blood or tumor sample, prior to and after the administration of a DNAPK inhibitor to said patient.

Also provided herein are methods for predicting the likelihood of a cancer of a patient being responsive to DNAPK inhibitor therapy, comprising: screening a biological sample of said patient for markers of Wnt activity, wherein the presence of markers of Wnt activity indicates an increased likelihood that a cancer of said patient will be responsive to DNAPK inhibitor therapy.

Further provided herein are methods for determining whether a patient is sensitive to a DNAPK inhibitor, comprising administering said patient said DNAPK inhibitor and determining whether markers of Wnt activity {[5-(3-fluorophenyl)-3-hydroxypyridine-2-carbonyl]amino}acetic acid are modulated in said patient by measuring the markers of Wnt activity in a biological sample from said patient, for example a peripheral blood or tumor sample, prior to and after the administration of the DNAPK inhibitor to said patient.

Also provided herein is a kit for detecting markers of Wnt activity comprising reagents for measuring markers of Wnt activity and one or more DNAPK inhibitors.

In some embodiments, the DNAPK inhibitor is a compound as described herein.

In some embodiments, the methods described herein, additionally comprise administration of a Wnt pathway modulator, a Wnt inhibitor, and/or an androgen receptor antagonist, as described herein.

The present embodiments can be understood more fully by reference to the detailed description and examples, which are intended to exemplify non-limiting embodiments.

### 4. DETAILED DESCRIPTION

### 4.1 Definitions

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturared alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl and the like. The double bond of an alkenyl group can be unconjugated or conjugated to another unsaturated group. An alkenyl group can be unsubstituted or substituted.

A “cycloalkyl” group is a saturated, or partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclylalkyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amine” group is a radical of the formula: —NH₂.

A “hydroxyl amine” group is a radical of the formula: —N(R^{#})OH or —NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: —N(R^{#})O-aryl or —NHO-aryl, wherein R^{#} is as defined above.

An “alkylamine” group is a radical of the formula: —NH-alkyl or —N(alkyl)₂, wherein each alkyl is independently as defined above.

An “aminocarbonyl” group is a radical of the formula: —C(═O)N(R^{#})₂, —C(═O)NH(R^{#}) or —C(═O)NH₂, wherein each R^{#} is as defined above.

An “acylamino” group is a radical of the formula: —NHC(═O)(R^{#}) or —N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O⁻.

A “carboxy” group is a radical of the formula: —C(═O)OH.

A “ketone” group is a radical of the formula: —C(═O)(R^{#}), wherein R^{#} is as defined above.

An “aldehyde” group is a radical of the formula: —CH(═O).

An “ester” group is a radical of the formula: —C(═O)O(R^{#}) or —OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or —NHC(═O)NH₂#, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R^{#})C(═O)(R^{#}) or —N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

A “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), —C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#})₂, —C(═N—NH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula: —N₃.

An “isocyanate” group is a radical of the formula: —N═C═O.

An “isothiocyanate” group is a radical of the formula: —N═C═S.

A “cyanate” group is a radical of the formula: —OCN.

A “thiocyanate” group is a radical of the formula: —SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, or —S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula: —P(R^{#})₂, wherein each R^{#} is independently as defined above.

When the groups described herein, with the exception of alkyl group are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the DNAPK inhibitors include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “clathrate” means a DNAPK inhibitor, or a salt thereof, in the form of a crystal lattice that contains spaces (e.g., channels) that have a guest molecule (e.g., a solvent or water) trapped within or a crystal lattice wherein a DNAPK inhibitor is a guest molecule.

As used herein and unless otherwise indicated, the term “solvate” means a DNAPK inhibitor, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a DNAPK inhibitor, or a salt thereof, that further includes a stoichiometric or non-stoichiometric amount of water bound by non-covalent intermolecular forces.

As used herein and unless otherwise indicated, the term “prodrug” means a DNAPK inhibitor derivative that can hydrolyze, oxidize, or otherwise react under biological conditions (in vitro or in vivo) to provide an active compound, particularly a DNAPK inhibitor. Examples of prodrugs include, but are not limited to, derivatives and metabolites of a DNAPK inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a DNAPK inhibitor that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The DNAPK inhibitors can have chiral centers and can occur as racemates, individual enantiomers or diastereomers, and mixtures thereof. All such isomeric forms are included within the embodiments disclosed herein, including mixtures thereof. The use of stereomerically pure forms of such DNAPK inhibitors, as well as the use of mixtures of those forms are encompassed by the embodiments disclosed herein. For example, mixtures comprising equal or unequal amounts of the enantiomers of a particular DNAPK inhibitor may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the DNAPK inhibitors can include E and Z isomers, or a mixture thereof, and cis and trans isomers or a mixture thereof. In certain embodiments, the DNAPK inhibitors are isolated as either the cis or trans isomer. In other embodiments, the DNAPK inhibitors are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

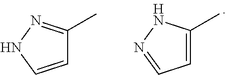

As readily understood by one skilled in the art, a wide variety of functional groups and other structures may exhibit tautomerism and all tautomers of the DNAPK inhibitors are within the scope of the present invention.

It should also be noted the DNAPK inhibitors can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the DNAPK inhibitors as described herein, whether radioactive or not, are intended to be encompassed within the scope of the embodiments provided herein. In some embodiments, there are provided isotopologues of the DNAPK inhibitors, for example, the isotopologues are deuterium, carbon-13, or nitrogen-15 enriched DNAPK inhibitors.

“Treating” as used herein, means an alleviation, in whole or in part, of a Wnt-associated cancer, or a symptom thereof, or slowing, or halting of further progression or worsening of a Wnt-associated cancer.

“Preventing” as used herein, means the prevention of the onset, recurrence or spread, in whole or in part, of a Wnt-associated cancer, or a symptom thereof.

The term “effective amount” in connection with an DNAPK inhibitor means an amount capable of alleviating, in whole or in part, symptoms associated with a Wnt-associated cancer, or slowing or halting further progression or worsening of those symptoms. The effective amount of the DNAPK inhibitor, for example in a pharmaceutical composition, may be at a level that will exercise the desired effect; for example, about 0.005 mg/kg of a subject's body weight to about 100 mg/kg of a patient's body weight in unit dosage for both oral and parenteral administration. As will be apparent to those skilled in the art, it is to be expected that the effective amount of a DNAPK inhibitor disclosed herein may vary depending on the severity of the indication being treated.

The terms “patient” and “subject” as used herein include an animal, including, but not limited to, an animal such as a cow, monkey, horse, sheep, pig, chicken, turkey, quail, cat, dog, mouse, rat, rabbit or guinea pig, in one embodiment a mammal, in another embodiment a human. In one embodiment, a “patient” or “subject” is a human having a Wnt-associated cancer.

“Wnt-associated cancer” refers to tumors in which Wnt signaling is dysregulated. This includes solid tumors (such as gastric cancer, breast cancer, endometrial cancer, uterine cancer, colorectal cancer, synovial sarcoma, pancreatic cancer, melanoma, lobular carcinoma, prostate cancer, triple negative breast cancer (TNBC), non-small cell lung cancer (NSCLC), squamous cell lung carcinoma, lung adenocarcinoma, hepatocellular cancer (HCC), ovarian cancer, adenoid carcinoma, adrenocortical carcinoma, bladder/urothelial carcinoma, glioblastoma multiforme (GBM), cervical cancer, head and neck squamous cell carcinoma (HNSCC), kidney cancer, and thyroid cancer) and hematologic malignancies (such as acute myelogenous leukemia (AML), acute lymphoblastic leukemia (ALL), multiple myeloma (MM), chronic lymphocytic leukemia (CLL), chronic myelogenous leukemia (CML)), as well as cancer stem cells in many tumors types, particularly those described herein.

“Wnt-inhibitors” refers to agents which reverse the dysregulated Wnt signaling in tumors and include downstream inhibitors of beta-catenin (niclosamide, XAV939, IWR, G0070-LK, Tautomycin, Pyrvinium, HQBA, PKF115-724, PKF115-584, PKF222-815, CGP049090, PRI-724, ICG001, AV65, JW55, G244-LM, WIKI4, iCRT3, iCRT5, iCRT14, 2,3 diamino-quinazoline, BC21, PNU-74654, curcumin, quercetin, RPI724, indirubins, bis-indoles, bio, DIF, Hexachlorophene, resveratrol), inhibitors of Wnt secretion (such as ETC-159, C59, IWP, LGK974), as well as recombinant proteins that decrease interactions of Wnt with their receptors (anti-Wnt antibodies, Foxy-5, sFRP, WIF1, anti-frizzled receptor antibodies (vantictumab), anti-RSPO3 antibodies, SOST, DKK, Fz decoy receptor fusion protein (OMP-54F28), FRZ8CRD, LRP inhibitors) or block aspects of Wnt signaling (such as NSC668036, 3289-8625, PCN-N3, FJ9, AV65, artificial F-Box, NSAIDs (such as sulindac, aspirin, celecoxib, rofecoxib, valdecoxib), thiazolidinedione antidiabetic agents (glitazones), AVI-4126, R-roscovitine (CYC202), rapamycin, or CCI-779).

“Wnt pathway modulators” include those which affect the hedgehog pathway (Smo antagonists (vismodegib, sonidegib, saridegib, BMS-833923, PF-04449913, LEQ506, TAK-441), Robotnikinin), the Notch pathways (mAbs to Notch ligands, notch decoys, mAbs to Notch receptors, g-secretase inhibitors, mABs to nicastrin), ABC transporters, chemotherapies (such as FOLFOX6, gemcitibine, dasatinib, cytarabine, paclitaxel, docetaxel, nab-paclitaxel, sorafenib, carboplatin or radiolabelled antibodies (such as OTSA101 (radiolabelled anti-Frizzled-10 antibody), and those which impact other signaling pathways, such as inhibitors of the Ras/Raf/MEK/ERK pathway, TGFb pathway, EGFR pathway (Tarceva, Iressa), PI3K/AKT/mTOR pathway, PPARγ (Troglitazone, rosiglitazone), PDGFR, KIT, Abl (STI-571, imatinib), retinoid X receptors (RXRs)/retinoic acid receptors (RARs) (such as 9-cis-RA, 4-HPR, IIF).

“Markers of Wnt activity” as used herein include mutations, copy number variations (CNV's, gains or losses), fusions, decreased/increased expression or mislocalization of miRNA, mRNA or protein, or changes in phosphorylation or activity of Wnt pathway genes or regulators (such as for example, Wnt ligands (including Wnt 1, 2, 2b, 3, 3a, 4, 5a, 5b, 6, 7a, 7b, 8a, 8b, 9a, 9b, 10a, 10b, 11, 16), Wnt receptors Frizzled's (Fzd 1, 2, 3, 4, 5, 6, 7, 8, 9, 10), LRP 5,6, APC, APC2, beta-catenin, GSK3α, GSK3β, β-TrCP, R-spondins RSPO1,2,3,4, LRP5/6, DVL1, DVL2, DVL3, EP300, FBXW11, FBXW2, FBXW4, FGF4, FOSL1, FOXN1, FRAT1, FRAT2, HDAC1, HPRT1, Jun, KREMEN1, CK1a, cmyc, GSKβ. AXIN1, AXIN2, c-myc, cyclin Dl, ACTB, AES, B2M, Bcl9, BTRC, CACYBP, CAMK2A, CAMK2B, CAMK2D, CAMK2G, CER1, CHD8, CHP2, CREBBP, CSNK1A1, CSNK1A1L, CSNK1D, CSNK1E, CSNK1G1, CSNK2A1, CSNK2A2, CSNK2B, CUL1, DAAM1, DAAM2, DIXDC1, DKK1, DKK2, DKK4, MAPK3K7, MAPK10, MAPK9, MMP7, YAP, TRIB2, HNF1A, PPARG, MMP7, CD44, COX2, LEF1, LEF2, sFRP1,2,4,5, WIF1, WIF2, Dkk-1,2,3, NKD1, Sox10, Sox17, HSulf1, RUNX3, PRDM5, RASSF10, OSR1, EZF1, HIPK1, RUNX2, PPN, DCH17, EZH2, HMGA1,2, YY1, TC1, CXXC4, TRF1, CPAP/CENP, plakoglobin, NuMA, IRAP, DACT1, DACT3, CTBP1, CTBP2, HNF4a, BTBC, CCND2, CCND3, TCF7L1, TCF7L2, TCF7, NFAT5, NFATC1, NFATC2, NFATC4, NKD2, NLK, PITX2, PLCB1, PLCB2, PLCB3, PLCB4, PORCN, PPARD, PPP2CA, PPP2CB, PPP2R1A, PPP2R1B, PPP2R5A, PPP2R5B, PPP2R5C, PPP2R5D, PPP2R5E, PPP3CA, PPP3CB, PPP3CC, PPP3R1, PPP3R2, PRICKLE1, PRICKLE2, PRKACB, PRKACG, PRKX, PSEN1, PSMA1-8, PSMB1-9, PSMC1-6, PSMD1-14, PSME1,2,4, PSMF1, PYGO1, RAC1,-3, RBX1, RHOA, RHOU, ROCK1,2, RPL13A, RPS27A, RUVBL1, SENP2, SFRP1,2,4,5, SIAH1, SKP1, SLC9A3R1, SMAD2,3,4, SOX17, T, TAB 1, TBL1, TBL1XR1, TLE1,2, TP53, UBA52, VANGL1, 2, WIF1, CTNNB1, CTNNBIP1ZNRF3, Notch1, Notch 2, Notch 3, Notch 4, Jagged 1, Jagged 2, numb, Gli1, TGFb, Sox2, Oct3/4, Klf4, Nanog, CDH1, CDH2, Zeb1, Zeb2, miR-17-92, Mir-10a, Has-miR-335, has-miR-375, miR-34c, miR-200c, miR203). Also included is the Wnt signature found via inhibition of Wnt (Ashihara, E. et al. Cancer Science vol 106, no 6, 665-671)), activation of other pathways (SMAD4 mutations, KRAS mutations) and markers (such as Sox7, RACK 1, ZNFR3, CDH8, PLA2GRA, Has-miR193b, miR 200a), which have been found to be associated with increased Wnt activity and/or response to inhibition of the Wnt pathway.

### 4.2 BRIEF DESCRIPTION OF THE DRAWINGS

FIG. 1 provides: Unbiased assessment of the prognostic potential of all kinases in the clinical cohort. The log 2 odds ratio of having a metastatic event with expression of every kinase is shown. Each kinase is represented by a bar. The enrichment of DNAPK is shown as the very left-most bar (panel A); Kaplan-Meier curves of clinical outcomes (metastasis-free survival, prostate cancer specific survival, and overall survival) by DNAPK low to high expression in discovery (cohort 1, n=545) and validation (cohort 2, n=130) cohorts (panel B); and Oncomine analysis of DNAPK expression in primary (left) vs metastatic (right) prostate cancer (panel C).

FIG. 2 provides: Representative images (200×) and crystal violet quantification of migration (panel A) and invasion (panel B) of C4-2, LNCaP-AR and PC3 cells in Boyden chamber assays after DNAPK knockdown (siRNA) or inhibition (NU7441); C4-2, LNCaP-AR and PC3 cell growth curves after DNAPK inhibition (NU7441) (panel C) or knockdown (siRNA) (panel D); and DNAPK knockdown by siRNA relative to non-targeting (siNTC) control (panel E). *p<0.05, **p<0.05 compared to respective controls. All graphs are mean±S.D.

FIG. 3 provides: Scatter plot of GSEA normalized enrichment scores (NES) for all pathways in the discovery clinical cohort (based on gene correlations with DNAPK) and in vitro knockdown of DNAPK (in VCaP, C4-2, PC3, DU145 cells; y axis). The gray datapoint for the Wnt signaling pathway is above the corresponding text (panel A); Expression of Wnt pathway target genes in CRPC cells LNCaP-AR and C4-2 compared to hormone-naive LNCaP cells (panel B); Growth curves of LNCaP cells under normal serum (black) or androgen-deprivation (charcoal-stripped serum, gray) conditions (panel C); Expression of Wnt pathway target genes in LNCaP cells grown in normal serum (black), or androgen deprivation (charcoal-stripped serum) conditions with control (siNTC, light gray) or DNAPK knockdown (siDNAPK, dark gray) (panel D); and Growth curves of androgen deprivation-resistant LNCaP cells (charcoal-stripped serum from panel C) switched to normal serum (black), or continued in androgen deprivation (charcoal-stripped serum) with control (siNTC, light gray) or DNAPK knockdown (siDNAPK, dark gray) (panel E). *=p<0.05, **p=<0.001. All graphs are mean±S.D. RQ=relative quantity. siNTC=non-targeting siRNA control.

FIG. 4 provides: Expression of Wnt pathway genes after DNAPK knockdown (panel A) or inhibition (NU7441) (panel B) in LNCaP-AR cells; Immunoblot analyses of DNAPK, phospho-DNAPK (pDNAPK), active beta catenin (βcatenin), and cMyc levels in LNCaP-AR and C4-2 cells after DNAPK knockdown (siDNAPK) or inhibition (NU7441) with or without Wnt3A stimulation (panels C-D); Representative images (200×) and crystal violet quantification of LNCaP-AR and C4-2 cell invasion and migration after DNAPK inhibition (NU7441 or Compound 1) with or without Wnt3A stimulation (panel E); and Representative images (200×) and crystal violet quantification of enzalutamide-resistant LNCaP-AR (LNCaP-AR-enza-res) cell invasion and migration after DNAPK inhibition (NU7441 or Compound 1) or continued enzalutamide treatment (Enza) (panel F). All graphs are mean±S.D. *=p<0.05 compared to respective controls. Enza=enzalutamide. Enza-res=enzalutamide resistant. RQ=relative quantity. siNTC=non-targeting siRNA control.

FIG. 5 provides: Immunoblot analyses of DNAPK, KU70 and LEF1 after DNAPK immunoprecipitation (left), and LEF1 and DNAPK after LEF1 immunoprecipitation (right) in LNCaP-AR cells (panel A); TOPFLASH luciferase reporter activity in PC3 cells after DNAPK inhibition (NU7441) and/or Wnt3A stimulation; activity is normalized to renilla luciferase (panel B); and Representative images (200×) and crystal violet quantification of LNCaP-AR and C4-2 cell invasion and migration after DNAPK, LEF1 or beta catenin (CTNNB1) knockdown by siRNA. *=p<0.05 compared to respective controls, #=p<0.05 compared to Wnt3A (panel C). All graphs are mean±S.D. siNTC=non targeting siRNA control.

FIG. 6 provides: Growth curves of LNCaP-AR xenografts in castrated mice treated daily with vehicle (black, n=5) or 25 mg/kg NU7441 (gray, n=6) (panel A); Fold change in final tumor volume (panel B); Kaplan-Meier curves of freedom from tumor doubling time with vehicle or NU7441 (25 mg/kg) treatment of LNCaP-AR xenograft tumors (panel C); Growth curves of LNCaP-AR xenograft tumors with 5 times weekly oral gavage enzalutamide (10 mg/kg, n=16), Compound 1 (2 mg/kg, n=14), Compound 1+enzalutamide (n=14), or vehicle (n=14) treatment. Bar graph represents change in final tumor volume compared to starting tumor volume (panel D); Kaplan-Meier curves of freedom from tumor tripling with enzalutamide, Compound 1, enzalutamide+Compound 1, or vehicle treatment of LNCaP-AR xenograft tumors (panel E); Expression of select Wnt pathway target genes in human primary prostate tumor explants treated with NU7441 (n=6) or vehicle (panel F); and Representative images of Ki67 immunostaining of human primary prostate tumor explants treated with NU7441 (n=6) or vehicle; Ki67-positive nuclei are indicated with arrows (panel G). All graphs are mean±S.E.M. *=p<0.05 compared to respective control, #=p<0.05 for enzalutamide compared to Compound 1+enzalutamide, **=p<0.01 for Compound 1 compared to vehicle.

FIG. 7 provides: Scatter plot showing correlation between DNAPK and 3 catenin (CTNNB1) expression in the discovery cohort (Clinical Cohort 1, n=545) (panel A); and Expression of Wnt pathway target genes in AR-independent PC3 cells relative to androgen-sensitive LNCaP cells (panel B). GAPDH was used as internal reference, bars are mean±S.D. RQ=relative quantity.

FIG. 8 provides: Expression of Wnt pathway genes in C4-2 cells after inhibition (NU7441) (panel A) or knockdown (panel B) of DNAPK; and Expression of Notch pathway genes in LNCaP-AR, PC3 and C4-2 cells after knockdown of DNAPK (panel C). GAPDH was used as internal reference, bars are mean±S.D. *=p<0.05. RQ=relative quantity.

FIG. 9 provides: Expression of Wnt pathway target genes in LNCaP-AR cells grown in enzalutamide until resistance (LNCaP-AR-enza-res) relative to LNCaP-AR cells grown in vehicle (DMSO) (panel A); and Expression of AR or AR-target genes FKBP5 and KLK3 (PSA) in LNCaP-AR-enza-res cells after treatment with 10 μM enzalutamide (panel B). GAPDH was used as internal reference, bars are mean±S.D. *=p<0.05. RQ=relative quantity.

FIG. 10 provides: Body weight measurements of LNCaP-AR xenograft mice after treatment with NU7441 (panel A); Growth curves of VCaP xenograft tumors with various doses of Compound 1 (administered once daily via oral gavage, 5 times per week for 6 weeks) or vehicle (panel B); Body weight measurements of VCaP xenograft mice after treatment with various Compound 1 doses (panel C); and Growth curves of PC3 xenograft tumors treated with Compound 1 or vehicle (panel D). All graphs are mean±S.E.M. *=p<0.05 compared to vehicle.

### 4.3 DNAPK INHIBITORS

The compounds provided herein are generally referred to as “DNAPK inhibitor(s).”

In one embodiment, the DNAPK inhibitors include compounds having the following formula (I):

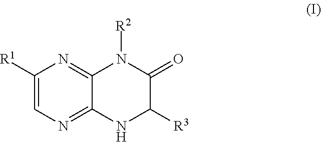

and pharmaceutically acceptable salts, clathrates, solvates, stereoisomers, tautomers, metabolites, isotopologues and prodrugs thereof, wherein:

R¹ is substituted or unsubstituted C₁₋₈ alkyl, substituted or unsubstituted aryl, substituted or unsubstituted cycloalkyl, substituted or unsubstituted heterocyclyl, or substituted or unsubstituted heterocyclylalkyl;

R² is H, substituted or unsubstituted C₁₋₈ alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl;

R³ is H, or a substituted or unsubstituted C₁₋₈ alkyl,

wherein in certain embodiments, the DNAPK inhibitors do not include 7-(4-hydroxyphenyl)-1-(3-methoxybenzyl)-3,4-dihydropyrazino[2,3-b]pyrazin-2(1H)-one, depicted below:

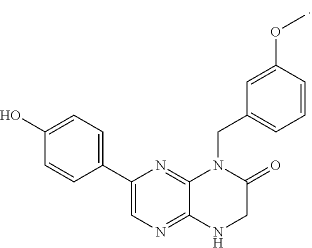 [SMILES: COc1cccc(CN2C(=O)CNc3ncc(-c4ccc(O)cc4)nc32)c1]

In some embodiments of compounds of formula (I), R¹ is substituted or unsubstituted aryl or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl or pyrazolyl), aminocarbonyl, halogen (for example, fluorine), cyano, hydroxyalkyl and hydroxy. In other embodiments, R¹ is pyridyl substituted with one or more substituents independently selected from the group consisting of substituted or unsubstituted C₁₋₈ alkyl (for example, methyl), substituted or unsubstituted heterocyclyl (for example, a substituted or unsubstituted triazolyl), halogen, aminocarbonyl, cyano, hydroxyalkyl (for example, hydroxypropyl), —OR, and —NR₂, wherein each R is independently H, or a substituted or unsubstituted C₁₋₄ alkyl. In some embodiments, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In some embodiments, R¹ is

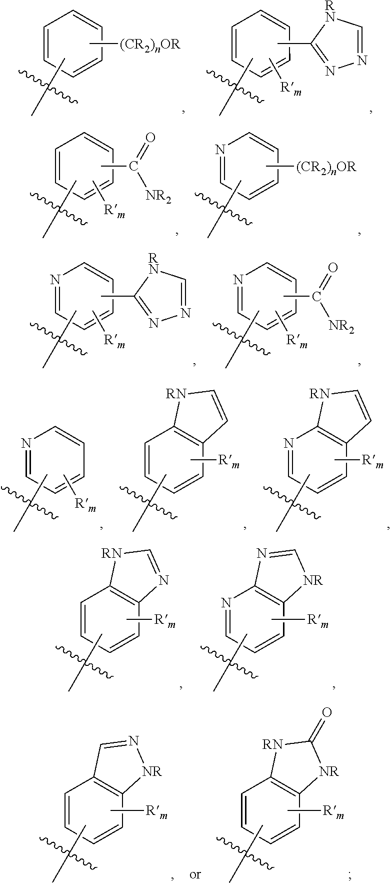

wherein R is at each occurrence independently H, or a substituted or unsubstituted C₁₋₄ alkyl (for example, methyl); R′ is at each occurrence independently a substituted or unsubstituted C₁₋₄ alkyl (for example, methyl), halogen (for example, fluoro), cyano, —OR, or —NR₂; m is 0-3; and n is 0-3. It will be understood by those skilled in the art that any of the substituents R′ may be attached to any suitable atom of any of the rings in the fused ring systems.

In some embodiments of compounds of formula (I), R¹ is

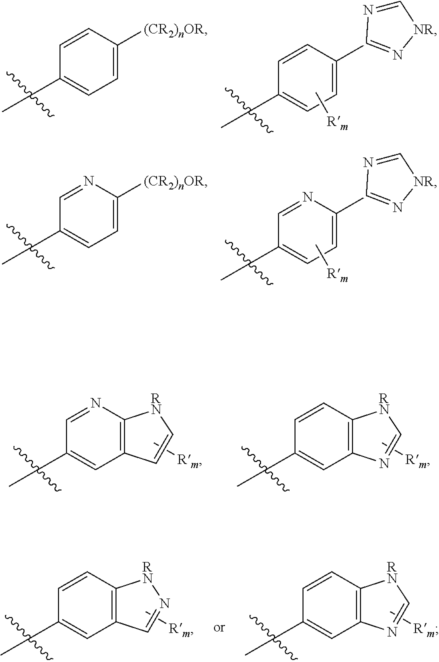

wherein R is at each occurrence independently H, or a substituted or unsubstituted C₁₋₄ alkyl; R′ is at each occurrence independently a substituted or unsubstituted C₁₋₄ alkyl, halogen, cyano, —OR or —NR₂; m is 0-3; and n is 0-3.

In some embodiments of compounds of formula (I), R² is H, substituted or unsubstituted C₁₋₈ alkyl, substituted or unsubstituted cycloalkyl, substituted or unsubstituted heterocyclyl, substituted or unsubstituted C₁₋₄ alkyl-heterocyclyl, substituted or unsubstituted C₁₋₄ alkyl-aryl, or substituted or unsubstituted C₁₋₄ alkyl-cycloalkyl. For example, R² is H, methyl, ethyl, n-propyl, isopropyl, n-butyl, sec-butyl, isobutyl, tert-butyl, n-pentyl, isopentyl, cyclopentyl, cyclohexyl, tetrahydrofuranyl, tetrahydropyranyl, (C₁₋₄ alkyl)-phenyl, (C₁₋₄ alkyl)-cyclopropyl, (C₁₋₄ alkyl)-cyclobutyl, (C₁₋₄ alkyl)-cyclopentyl, (C₁₋₄ alkyl)-cyclohexyl, (C₁₋₄ alkyl)-pyrrolidyl, (C₁₋₄ alkyl)-piperidyl, (C₁₋₄ alkyl)-piperazinyl, (C₁₋₄ alkyl)-morpholinyl, (C₁₋₄ alkyl)-tetrahydrofuranyl, or (C₁₋₄ alkyl)-tetrahydropyranyl, each optionally substituted.

In other embodiments, R² is H, C₁₋₄ alkyl, (C₁₋₄alkyl)(OR),

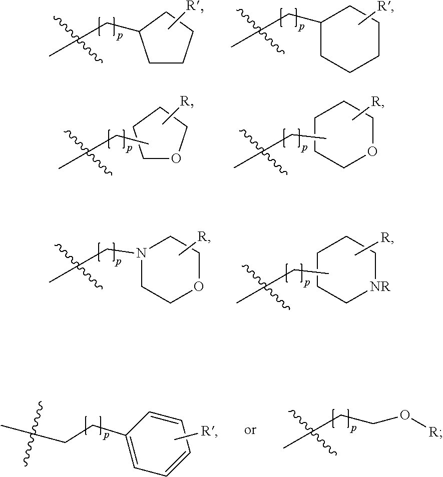

wherein R is at each occurrence independently H, or a substituted or unsubstituted C₁₋₄ alkyl (for example, methyl); R′ is at each occurrence independently H, —OR, cyano, or a substituted or unsubstituted C₁₋₄ alkyl (for example, methyl); and p is 0-3.

In other embodiments of compounds of formula (I), R² is H, C₁₋₄ alkyl, (C₁₋₄alkyl)(OR),

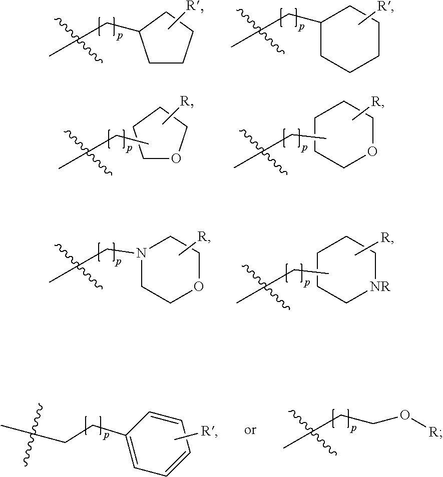

wherein R is at each occurrence independently H, or a substituted or unsubstituted C₁₋₂ alkyl; R′ is at each occurrence independently H, —OR, cyano, or a substituted or unsubstituted C₁₋₂ alkyl; and p is 0-1.

In other embodiments of compounds of formula (I), R³ is H.

In some such embodiments described herein, R¹ is substituted or unsubstituted aryl, or substituted or unsubstituted heteroaryl. For example, R¹ is phenyl, pyridyl, pyrimidyl, benzimidazolyl, 1H-pyrrolo[2,3-b]pyridyl, indazolyl, indolyl, 1H-imidazo[4,5-b]pyridine, pyridyl, 1H-imidazo[4,5-b]pyridin-2(3H)-onyl, 3H-imidazo[4,5-b]pyridyl, or pyrazolyl, each optionally substituted. In some embodiments, R¹ is phenyl substituted with one or more substituents independently selected from the group consisting of substituted or unsubstituted C₁₋₈ alkyl, substituted or unsubstituted heterocyclyl, aminocarbonyl, halogen, cyano, hydroxyalkyl and hydroxy. In others, R¹ is pyridyl substituted with one or more substituents independently selected from the group consisting of C₁₋₈ alkyl, substituted or unsubstituted heterocyclyl, halogen, aminocarbonyl, cyano, hydroxyalkyl, —OR, and —NR₂, wherein each R is independently H, or a substituted or unsubstituted C₁₋₄ alkyl. In still others, R¹ is 1H-pyrrolo[2,3-b]pyridyl or benzimidazolyl, optionally substituted with one or more substituents independently selected from the group consisting of substituted or unsubstituted C₁₋₈ alkyl, and —NR₂, wherein R is independently H, or a substituted or unsubstituted C₁₋₄ alkyl.

In certain embodiments, the compounds of formula (I) have an R¹ group set forth herein and an R² group set forth herein.

In some embodiments of compounds of formula (I), the compound inhibits DNAPK.

In some embodiments of compounds of formula (I), the compound at a concentration of 10 μM inhibits DNAPK by at least about 50%. Compounds of formula (I) may be shown to be inhibitors of DNAPK in any suitable assay system.

Representative DNAPK inhibitors of formula (I) include compounds from Table A.

TABLE A
7-(5-fluoro-2-methyl-4-(1H-1,2,4-triazol-3-yl)phenyl)-1-((trans-4-methoxycyclohexyl)methyl)-
3,4-dihydropyrazino[2,3-b]pyrazin-2(1H)-one;
7-(6-(1H-1,2,4-triazol-3-yl)pyridin-3-yl)-1-(cis-4-methoxycyclohexyl)-3,4-
dihydropyrazino[2,3-b]pyrazin-2(1H)-one;
7-(1H-pyrrolo[2,3-b]pyridin-3-yl)-1-(2-(tetrahydro-2H-pyran-4-yl)ethyl)-3,4-
dihydropyrazino[2,3-b]pyrazin-2(1H)-one;
7-(5-fluoro-2-methyl-4-(1H-1,2,4-triazol-3-yl)phenyl)-1-((cis-4-methoxycyclohexyl)methyl)-
3,4-dihydropyrazino[2,3-b]pyrazin-2(1H)-one;
1-ethyl-7-(1H-pyrrolo[3,2-b]pyridin-5-yl)-3,4-dihydropyrazino[2,3-b]pyrazin-2(1H)-one;
7-(6-(1H-1,2,4-triazol-3-yl)pyridin-3-yl)-1-((cis-4-methoxycyclohexyl)methyl)-3,4-
dihydropyrazino[2,3-b]pyrazin-2(1H)-one;
7-(1H-benzo[d]imidazol-4-yl)-1-(2-(tetrahydro-2H-pyran-4-yl)ethyl)-3,4-dihydropyrazino[2,3-
b]pyrazin-2(1H)-one;
7-(1H-pyrrolo[2,3-b]pyridin-4-yl)-1-(2-(tetrahydro-2H-pyran-4-yl)ethyl)-3,4-
dihydropyrazino[2,3-b]pyrazin-2(1H)-one;
7-(6-(1H-1,2,4-triazol-3-yl)pyridin-3-yl)-1-((trans-4-methoxycyclohexyl)methyl)-3,4-
dihydropyrazino[2,3-b]pyrazin-2(1H)-one;
7-(6-(1H-1,2,4-triazol-3-yl)pyridin-3-yl)-1-((trans-4-hydroxycyclohexyl)methyl)-3,4-
dihydropyrazino[2,3-b]pyrazin-2(1H)-one;
7-(6-(1H-1,2,4-triazol-3-yl)pyridin-3-yl)-1-(cis-4-hydroxycyclohexyl)-3,4-dihydropyrazino[2,3-
b]pyrazin-2(1H)-one;
7-(5-fluoro-2-methyl-4-(1H-1,2,4-triazol-3-yl)phenyl)-1-(cis-4-hydroxycyclohexyl)-3,4-
dihydropyrazino[2,3-b]pyrazin-2(1H)-one;
7-(6-(1H-1,2,4-triazol-3-yl)pyridin-3-yl)-1-(tetrahydro-2H-pyran-4-yl)-3,4-
dihydropyrazino[2,3-bl]pyrazin-2(1H)-one;
7-(6-(1H-1,2,4-triazol-3-yl)pyridin-3-yl)-1-(2-methoxyethyl)-3,4-dihydropyrazino[2,3-
b]pyrazin-2(1H)-one;
7-(6-(1H-1,2,4-triazol-3-yl)pyridin-3-yl)-1-ethyl-3,4-dihydropyrazino[2,3-b]pyrazin-2(1H)-one;
7-(5-fluoro-2-methyl-4-(1H-1,2,4-triazol-3-yl)phenyl)-1-((cis-4-hydroxycyclohexyl)methyl)-
3,4-dihydropyrazino[2,3-b]pyrazin-2(1H)-one;
7-(5-fluoro-2-methyl-4-(1H-1,2,4-triazol-3-yl)phenyl)-1-(tetrahydro-2H-pyran-4-yl)-3,4-
dihydropyrazino[2,3-b]pyrazin-2(1H)-one;
7-(1H-indol-4-yl)-1-(2-(tetrahydro-2H-pyran-4-yl)ethyl)-3,4-dihydropyrazino[2,3-b]pyrazin-
2(1H)-one;
7-(5-fluoro-2-methyl-4-(1H-1,2,4-triazol-3-yl)phenyl)-1-((trans-4-hydroxycyclohexyl)methyl)-
3,4-dihydropyrazino[2,3-b]pyrazin-2(1H)-one;
7-(6-(1H-1,2,4-triazol-3-yl)pyridin-3-yl)-1-((cis-4-hydroxycyclohexyl)methyl)-3,4-
dihydropyrazino[2,3-b]pyrazin-2(1H)-one;
7-(6-(1H-1,2,4-triazol-3-yl)pyridin-3-yl)-1-(trans-4-hydroxycyclohexyl)-3,4-
dihydropyrazino[2,3-b]pyrazin-2(1H)-one;
7-(6-(1H-1,2,4-triazol-3-yl)pyridin-3-yl)-1-(trans-4-methoxycyclohexyl)-3,4-
dihydropyrazino[2,3-b]pyrazin-2(1H)-one;
7-(6-(1H-1,2,4-triazol-3-yl)pyridin-3-yl)-1-isopropyl-3,4-dihydropyrazino[2,3-b]pyrazin-2(1H)-
one;
7-(5-fluoro-2-methyl-4-(1H-1,2,4-triazol-3-yl)phenyl)-1-(trans-4-methoxycyclohexyl)-3,4-
dihydropyrazino[2,3-b]pyrazin-2(1H)-one;
7-(5-fluoro-2-methyl-4-(1H-1,2,4-triazol-3-yl)phenyl)-1-(trans-4-hydroxycyclohexyl)-3,4-
dihydropyrazino[2,3-b]pyrazin-2(1H)-one;
7-(5-fluoro-2-methyl-4-(1H-1,2,4-triazol-3-yl)phenyl)-1-(2-methoxyethyl)-3,4-
dihydropyrazino[2,3-b]pyrazin-2(1H)-one;
7-(5-fluoro-2-methyl-4-(1H-1,2,4-triazol-3-yl)phenyl)-1-isopropyl-3,4-dihydropyrazino[2,3-
b]pyrazin-2(1H)-one;
1-ethyl-7-(5-fluoro-2-methyl-4-(1H-1,2,4-triazol-3-yl)phenyl)-3,4-dihydropyrazino[2,3-
b]pyrazin-2(1H)-one;
7-(2-hydroxypyridin-4-yl)-1-(2-(tetrahydro-2H-pyran-4-yl)ethyl)-3,4-dihydropyrazino[2,3-
b]pyrazin-2(1H)-one;
1-isopropyl-7-(4-methyl-6-(1H-1,2,4-triazol-3-yl)pyridin-3-yl)-3,4-dihydropyrazino[2,3-
b]pyrazin-2(1H)-one;
5-(8-isopropyl-7-oxo-5,6,7,8-tetrahydropyrazino[2,3-b]pyrazin-2-yl)-4-methylpicolinamide;
7-(1H-indazol-4-yl)-1-(2-(tetrahydro-2H-pyran-4-yl)ethyl)-3,4-dihydropyrazino[2,3-b]pyrazin-
2(1H)-one;
7-(2-aminopyrimidin-5-yl)-1-(2-(tetrahydro-2H-pyran-4-yl)ethyl)-3,4-dihydropyrazino[2,3-
b]pyrazin-2(1H)-one;
7-(2-aminopyridin-4-yl)-1-(2-(tetrahydro-2H-pyran-4-yl)ethyl)-3,4-dihydropyrazino[2,3-
b]pyrazin-2(1H)-one;
7-(6-(methylamino)pyridin-3-yl)-1-(2-(tetrahydro-2H-pyran-4-yl)ethyl)-3,4-
dihydropyrazino[2,3-b]pyrazin-2(1H)-one;
7-(6-hydroxypyridin-3-yl)-1-(2-(tetrahydro-2H-pyran-4-yl)ethyl)-3,4-dihydropyrazino[2,3-
b]pyrazin-2(1H)-one;
7-(4-(1H-pyrazol-3-yl)phenyl)-1-(2-methoxyethyl)-3,4-dihydropyrazino[2,3-b]pyrazin-2(1H)-
one;
7-(pyridin-3-yl)-1-(2-(tetrahydro-2H-pyran-4-yl)ethyl)-3,4-dihydropyrazino[2,3-b]pyrazin-
2(1H)-one;
7-(1H-indazol-4-yl)-1-(2-methoxyethyl)-3,4-dihydropyrazino[2,3-b]pyrazin-2(1H)-one;
7-(1H-indazol-6-yl)-1-(2-methoxyethyl)-3,4-dihydropyrazino[2,3-b]pyrazin-2(1H)-one;
7-(pyrimidin-5-yl)-1-(2-(tetrahydro-2H-pyran-4-yl)ethyl)-3,4-dihydropyrazino[2,3-b]pyrazin-
2(1H)-one;
7-(6-methoxypyridin-3-yl)-1-(2-(tetrahydro-2H-pyran-4-yl)ethyl)-3,4-dihydropyrazino[2,3-
b]pyrazin-2(1H)-one;
1-(2-methoxyethyl)-7-(1H-pyrrolo[2,3-b]pyridin-5-yl)-3,4-dihydropyrazino[2,3-b]pyrazin-
2(1H)-one;
1-ethyl-7-(1H-pyrrolo[2,3-b]pyridin-5-yl)-3,4-dihydropyrazino[2,3-b]pyrazin-2(1H)-one;
1-ethyl-7-(1H-indazol-4-yl)-3,4-dihydropyrazino[2,3-b]pyrazin-2(1H)-one;
7-(pyridin-4-yl)-1-(2-(tetrahydro-2H-pyran-4-yl)ethyl)-3,4-dihydropyrazino[2,3-b]pyrazin-
2(1H)-one;
7-(6-aminopyridin-3-yl)-1-(2-(tetrahydro-2H-pyran-4-yl)ethyl)-3,4-dihydropyrazino[2,3-
b]pyrazin-2(1H)-one;
l-methyl-7-(2-methyl-6-(4H-1,2,4-triazol-3-yl)pyridin-3-yl)-3,4-dihydropyrazino[2,3-
b]pyrazin-2(1H)-one;
2-(2-hydroxypropan-2-yl)-5-(8-(trans-4-methoxycyclohexyl)-7-oxo-5,6,7,8-
tetrahydropyrazino[2,3-b]pyrazin-2-yl)pyridine 1-oxide;
4-methyl-5-(7-oxo-8-((tetrahydro-2H-pyran-4-yl)methyl)-5,6,7,8-tetrahydropyrazino[2,3-
b]pyrazin-2-yl)picolinamide;
5-(8-((cis-4-methoxycyclohexyl)methyl)-7-oxo-5,6,7,8-tetrahydropyrazino[2,3-b]pyrazin-2-yl)-
4-methylpicolinamide;
7-(1H-pyrazol-4-yl)-1-(2-(tetrahydro-2H-pyran-4-yl)ethyl)-3,4-dihydropyrazino[2,3-b]pyrazin-
2(1H)-one;
1-(trans-4-methoxycyclohexyl)-7-(4-methyl-6-(1H-1,2,4-triazol-3-yl)pyridin-3-yl)-3,4-
dihydropyrazino[2,3-b]pyrazin-2(1H)-one;
3-((7-(2-methyl-6-(4H-1,2,4-triazol-3-yl)pyridin-3-yl)-2-oxo-3,4-dihydropyrazino[2,3-
b]pyrazin-1(2H)-yl)methyl)benzonitrile;
1-((trans-4-methoxycyclohexyl)methyl)-7-(4-methyl-6-(1H-1,2,4-triazol-3-yl)pyridin-3-yl)-3,4-
dihydropyrazino[2,3-b]pyrazin-2(1H)-one;
3-(7-oxo-8-(2-(tetrahydro-2H-pyran-4-yl)ethyl)-5,6,7,8-tetrahydropyrazino[2,3-b]pyrazin-2-
yl)benzamide;
5-(8-((trans-4-methoxycyclohexyl)methyl)-7-oxo-5,6,7,8-tetrahydropyrazino[2,3-b]pyrazin-2-
yl)-4-methylpicolinamide;
3-((7-(6-(2-hydroxypropan-2-yl)pyridin-3-yl)-2-oxo-3,4-dihydropyrazino[2,3-b]pyrazin-1(2H)-
yl)methyl)benzonitrile;
7-(6-(2-hydroxypropan-2-yl)pyridin-3-yl)-1-((1R,3R)-3-methoxycyclopentyl)-3,4-
dihydropyrazino[2,3-b]pyrazin-2(1H)-one;
7-(6-(2-hydroxypropan-2-yl)pyridin-3-yl)-1-((1S,3R)-3-methoxycyclopentyl)-3,4-
dihydropyrazino[2,3-b]pyrazin-2(1H)-one;
7-(6-(2-hydroxypropan-2-yl)pyridin-3-yl)-1-((1S,3S)-3-methoxycyclopentyl)-3,4-
dihydropyrazino[2,3-b]pyrazin-2(1H)-one;
7-(6-(2-hydroxypropan-2-yl)pyridin-3-yl)-1-((1R,3S)-3-methoxycyclopentyl)-3,4-
dihydropyrazino[2,3-b]pyrazin-2(1H)-one;
7-(1H-indazol-6-yl)-1-(2-(tetrahydro-2H-pyran-4-yl)ethyl)-3,4-dihydropyrazino[2,3-b]pyrazin-
2(1H)-one;
7-(2-methyl-6-(4H-1,2,4-triazol-3-yl)pyridin-3-yl)-1-(2-morpholinoethyl)-3,4-
dihydropyrazino[2,3-b]pyrazin-2(1H)-one;
1-(trans-4-hydroxycyclohexyl)-7-(2-methyl-6-(4H-1,2,4-triazol-3-yl)pyridin-3-yl)-3,4-
dihydropyrazino[2,3-b]pyrazin-2(1H)-one;
1-(cis-4-hydroxycyclohexyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-(2-morpholinoethyl)-3,4-dihydropyrazino[2,3-
b]pyrazin-2(1H)-one;
1-isopropyl-7-(2-methyl-6-(4H-1,2,4-triazol-3-yl)pyridin-3-yl)-3,4-dihydropyrazino[2,3-
b]pyrazin-2(1H)-one;
7-(1H-imidazo[4,5-b]pyridin-6-yl)-1-(2-(tetrahydro-2H-pyran-4-yl)ethyl)-3,4-
dihydropyrazino[2,3-b]pyrazin-2(1H)-one;
1-((cis-4-methoxycyclohexyl)methyl)-7-(2-methyl-6-(1H-1,2,4-triazol-3-yl)pyridin-3-yl)-3,4-
dihydropyrazino[2,3-b]pyrazin-2(1H)-one;
1-(trans-4-hydroxycyclohexyl)-7-(6-(2-hydroxypropan-2-yl)pyridin-3-yl)-3,4-
dihydropyrazino[2,3-b]pyrazin-2(1H)-one;
1-(cis-4-hydroxycyclohexyl)-7-(6-(2-hydroxypropan-2-yl)pyridin-3-yl)-3,4-
dihydropyrazino[2,3-b]pyrazin-2(1H)-one;
4-(7-oxo-8-(2-(tetrahydro-2H-pyran-4-yl)ethyl)-5,6,7,8-tetrahydropyrazino[2,3-b]pyrazin-2-
yl)benzamide;
7-(1H-indazol-5-yl)-1-(2-(tetrahydro-2H-pyran-4-yl)ethyl)-3,4-dihydropyrazino[2,3-b]pyrazin-
2(1H)-one;
7-(1H-pyrrolo[2,3-b]pyridin-5-yl)-1-(2-(tetrahydro-2H-pyran-4-yl)ethyl)-3,4-
dihydropyrazino[2,3-b]pyrazin-2(1H)-one;
7-(2-methyl-6-(4H-1,2,4-triazol-3-yl)pyridin-3-yl)-1-(tetrahydro-2H-pyran-4-yl)-3,4-
dihydropyrazino[2,3-b]pyrazin-2(1H)-one;
1-((1S,3R)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1R,3R)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1R,3S)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
1-((1S,3S)-3-methoxycyclopentyl)-7-(2-methyl-6-(4H-1,2,4-triazol-3-yl)pyridin-3-yl)-3,4-
dihydropyrazino[2,3-b]pyrazin-2(1H)-one;
7-(1H-indol-5-yl)-1-(2-(tetrahydro-2H-pyran-4-yl)ethyl)-3,4-dihydropyrazino[2,3-b]pyrazin-
2(1H)-one;
1-ethyl-7-(2-methyl-6-(4H-1,2,4-triazol-3-yl)pyridin-3-yl)-3,4-dihydropyrazino[2,3-b]pyrazin-
2(1H)-one;
7-(1H-indol-6-yl)-1-(2-(tetrahydro-2H-pyran-4-yl)ethyl)-3,4-dihydropyrazino[2,3-b]pyrazin-
2(1H)-one;
7-(4-(2-hydroxypropan-2-yl)phenyl)-1-(trans-4-methoxycyclohexyl)-3,4-dihydropyrazino[2,3-
b]pyrazin-2(1H)-one;
7-(6-(2-hydroxypropan-2-yl)pyridin-3-yl)-1-(tetrahydro-2H-pyran-4-yl)-3,4-
dihydropyrazino[2,3-b]pyrazin-2(1H)-one;
1-((trans-4-methoxycyclohexyl)methyl)-7-(2-methyl-6-(1H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-((cis-4-methoxycyclohexyl)methyl)-3,4-
dihydropyrazino[2,3-b]pyrazin-2(1H)-one;
1-(2-methoxyethyl)-7-(4-methyl-2-(methylamino)-1H-benzo[d]imidazol-6-yl)-3,4-
dihydropyrazino[2,3-b]pyrazin-2(1H)-one;
7-(7-methyl-2-oxo-2,3-dihydro-1H-benzo[d]imidazol-5-yl)-1-((tetrahydro-2H-pyran-4-
yl)methyl)-3,4-dihydropyrazino[2,3-b]pyrazin-2(1H)-one;
7-(2-methyl-4-(4H-1,2,4-triazol-3-yl)phenyl)-3,4-dihydropyrazino[2,3-b]pyrazin-2(1H)-one;
1-(2-methoxyethyl)-7-(4-methyl-6-(1H-1,2,4-triazol-3-yl)pyridin-3-yl)-3,4-
dihydropyrazino[2,3-b]pyrazin-2(1H)-one;
l-benzyl-7-(2-methyl-4-(4H-1,2,4-triazol-3-yl)phenyl)-3,4-dihydropyrazino[2,3-b]pyrazin-
2(1H)-one;
7-(3-fluoro-4-(4H-1,2,4-triazol-3-yl)phenyl)-1-(2-methoxyethyl)-3,4-dihydropyrazino[2,3-
b]pyrazin-2(1H)-one;
7-(3-fluoro-4-(4H-1,2,4-triazol-3-yl)phenyl)-1-(2-(tetrahydro-2H-pyran-4-yl)ethyl)-3,4-
dihydropyrazino[2,3-b]pyrazin-2(1H)-one;
7-(3-fluoro-2-methyl-4-(1H-1,2,4-triazol-3-yl)phenyl)-1-(2-methoxyethyl)-3,4-
dihydropyrazino[2,3-b]pyrazin-2(1H)-one;
1-(trans-4-methoxycyclohexyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-(trans-4-rnethoxycyclohexyl)-3,4-
dihydropyrazino[2,3-b]pyrazin-2(1H)-one;
7-(5-fluoro-2-methyl-4-(4H-1,2,4-triazol-3-yl)phenyl)-1-(2-(tetrahydro-2H-pyran-4-yl)ethyl)-
3,4-dihydropyrazino[2,3-b]pyrazin-2(1H)-one;
7-(3-fluoro-2-methyl-4-(1H-1,2,4-triazol-3-yl)phenyl)-1-(2-(tetrahydro-2H-pyran-4-yl)ethyl)-
3,4-dihydropyrazino[2,3-b]pyrazin-2(1H)-one;
1-(2-methoxyethyl)-7-(2-methyl-6-(4H-1,2,4-triazol-3-yl)pyridin-3-yl)-3,4-
dihydropyrazino[2,3-b]pyrazin-2(1H)-one;
7-(6-(2-hydroxypropan-2-yl)pyridin-3-yl)-1-((trans-4-methoxycyclohexyl)methyl)-3,4-
dihydropyrazino[2,3-b]pyrazin-2(1H)-one;
1-(cyclopentylmethyl)-7-(6-(2-hydroxypropan-2-yl)pyridin-3-yl)-3,4-dihydropyrazino[2,3-
b]pyrazin-2(1H)-one;
7-(4-(2-hydroxypropan-2-yl)phenyl)-1-(2-methoxyethyl)-3,4-dihydropyrazino[2,3-b]pyrazin-
2(1H)-one;
(S)-7-(6-(1-hydroxyethyl)pyridin-3-yl)-1-(2-(tetrahydro-2H-pyran-4-yl)ethyl)-3,4-
dihydropyrazino[2,3-b]pyrazin-2(1H)-one;
(R)-7-(6-(1-hydroxyethyl)pyridin-3-yl)-1-(2-(tetrahydro-2H-pyran-4-yl)ethyl)-3,4-
dihydropyrazino[2,3-b]pyrazin-2(1H)-one;
7-(2-methyl-6-(4H-1,2,4-triazol-3-yl)pyridin-3-yl)-1-((tetrahydro-2H-pyran-4-yl)methyl)-3,4-
dihydropyrazino[2,3-b]pyrazin-2(1H)-one;
7-(4-(2-hydroxypropan-2-yl)phenyl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(4-(trifluoromethyl)benzyl)-3,4-
dihydropyrazino[2,3-b]pyrazin-2(1H)-one;
7-(6-(2-hydroxypropan-2-yl)pyridin-3-yl)-1-(3-(trifluoromethyl)benzyl)-3,4-
dihydropyrazino[2,3-b]pyrazin-2(1H)-one;
7-(6-(2-hydroxypropan-2-yl)pyridin-3-yl)-1-(3-methoxypropyl)-3,4-dihydropyrazino[2,3-
b]pyrazin-2(1H)-one;
7-(4-methyl-6-(1H-1,2,4-triazol-3-yl)pyridin-3-yl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(2-methoxyethyl)-3,4-dihydropyrazino[2,3-
b]pyrazin-2(1H)-one;
7-(6-(2-hydroxypropan-2-yl)pyridin-3-yl)-1-((tetrahydro-2H-pyran-4-yl)methyl)-3,4-
dihydropyrazino[2,3-b]pyrazin-2(1H)-one;
7-(4-methyl-2-(methylamino)-1H-benzo[d]imidazol-6-yl)-1-((tetrahydro-2H-pyran-4-
yl)methyl)-3,4-dihydropyrazino[2,3-b]pyrazin-2(1H)-one;
7-(2-amino-4-methyl-1H-benzo[d]imidazol-6-yl)-1-((tetrahydro-2H-pyran-4-yl)methyl)-3,4-
dihydropyrazino[2,3-b]pyrazin-2(1H)-one;
7-(2-methyl-6-(4H-1,2,4-triazol-3-yl)pyridin-3-yl)-1-(2-(tetrahydro-2H-pyran-4-yl)ethyl)-3,4-
dihydropyrazino[2,3-b]pyrazin-2(1H)-one;
(R)-7-(6-(2-hydroxypropan-2-yl)pyridin-3-yl)-3-methyl-1-(2-(tetrahydro-2H-pyran-4-yl)ethyl)-
3,4-dihydropyrazino[2,3-b]pyrazin-2(1H)-one;
(S)-7-(6-(2-hydroxypropan-2-yl)pyridin-3-yl)-3-methyl-1-(2-(tetrahydro-2H-pyran-4-yl)ethyl)-
3,4-dihydropyrazino[2,3-b]pyrazin-2(1H)-one;
7-(6-(2-hydroxypropan-2-yl)pyridin-3-yl)-3,3-dimethyl-1-(2-(tetrahydro-2H-pyran-4-yl)ethyl)-
3,4-dihydropyrazino[2,3-b]pyrazin-2(1H)-one;
7-(2-amino-4-methyl-1H-benzo[d]imidazol-6-yl)-1-(2-(tetrahydro-2H-pyran-4-yl)ethyl)-3,4-
dihydropyrazino[2,3-b]pyrazin-2(1H)-one;
7-(6-(2-hydroxypropan-2-yl)pyridin-3-yl)-1-(2-(tetrahydro-2H-pyran-4-yl)ethyl)-3,4-
dihydropyrazino[2,3-b]pyrazin-2(1H)-one;
7-(2-methyl-4-(1H-1,2,4-triazol-3-yl)phenyl)-1-(2-(tetrahydro-2H-pyran-4-yl)ethyl)-3,4-
dihydropyrazino[2,3-b]pyrazin-2(1H)-one;
7-(4-(1H-1,2,4-triazol-5-yl)phenyl)-1-(2-(tetrahydro-2H-pyran-4-yl)ethyl)-3,4-
dihydropyrazino[2,3-b]pyrazin-2(1H)-one;
1-(1-hydroxypropan-2-yl)-7-(2-methyl-6-(1H-1,2,4-triazol-3-yl)pyridin-3-yl)-3,4-
dihydropyrazino[2,3-b]pyrazin-2(1H)-one; and
1-(2-hydroxyethyl)-7-(2-methyl-6-(1H-1,2,4-triazol-3-yl)pyridin-3-yl)-3,4-
dihydropyrazino[2,3-b]pyrazin-2(1H)-one,
and pharmaceutically acceptable salts, clathrates, solvates, stereoisomers, tautomers,
metabolites, isotopologues and prodrugs thereof.

In one embodiment, Compound 1 is 1-ethyl-7-(2-methyl-6-(1H-1,2,4-triazol-3-yl)pyridin-3-yl)-3,4-dihydropyrazino[2,3-b]pyrazin-2(1H)-one, or a tautomer thereof, for example, 1-ethyl-7-(2-methyl-6-(4H-1,2,4-triazol-3-yl)pyridin-3-yl)-3,4-dihydropyrazino[2,3-b]pyrazin-2(1H)-one, or 1-ethyl-7-(2-methyl-6-(1H-1,2,4-triazol-5-yl)pyridin-3-yl)-3,4-dihydropyrazino[2,3-b]pyrazin-2(1H)-one.

### 4.4 Methods for Making DNAPK Inhibitors

The DNAPK inhibitors can be obtained via standard, well-known synthetic methodology, see e.g., March, J. Advanced Organic Chemistry; Reactions Mechanisms, and Structure, 4th ed., 1992. Starting materials useful for preparing compounds of formula (III) and intermediates therefore, are commercially available or can be prepared from commercially available materials using known synthetic methods and reagents.

Particular methods for preparing compounds of formula (I) are disclosed in U.S. Pat. No. 8,110,578, issued Feb. 7, 2012, and U.S. Pat. No. 8,569,494, issued Oct. 29, 2013, each incorporated by reference herein in their entirety.

### 4.5 Methods of Use

Provided herein are methods for treating or preventing Wnt-associated cancers, comprising administering an effective amount of a DNAPK inhibitor to a patient having a Wnt-associated cancer as well as the use of a DNAPK-inhibitor in methods for treating or preventing Wnt-associated cancers.

Further provided herein are methods for inhibiting or preventing metastasis of Wnt-associated cancers, comprising administering an effective amount of a DNAPK inhibitor to a patient having a Wnt-associated cancer.

Further provided herein are methods for inhibiting or preventing expansion or survival of cancer stem cells of Wnt-associated cancers, comprising contacting the cancer stem cells of a Wnt-associated cancer with an effective amount of a DNAPK inhibitor. In certain embodiments, the contacting of a cancer stem cell of a Wnt-associated cancer with an effective amount of a DNAPK inhibitor is achieved by administering a DNAPK inhibitor to a patient having a Wnt-associated cancer. In other embodiments, the contacting of a cancer stem cell of a Wnt-associated cancer with an effective amount of a DNAPK inhibitor is achieved by contacting a biological sample (e.g., a tumor, blood or tissue sample) of a patient having a Wnt-associated cancer ex vivo with a DNAPK inhibitor.

Further provided herein are methods for inhibiting or preventing expansion or survival of resistant and/or refractory tumor cells of Wnt-associated cancers, comprising contacting the tumor cells of the Wnt-associated cancer with an effective amount of a DNAPK inhibitor. In certain embodiments, the contacting of a resistant and/or refractory tumor cell of a Wnt-associated cancer with an effective amount of a DNAPK inhibitor is achieved by administering a DNAPK inhibitor to a patient having a resistant and/or refractory Wnt-associated cancer. In other embodiments, the contacting of a resistant and/or refractory tumor cell of Wnt-associated cancer with an effective amount of a DNAPK inhibitor is achieved by contacting a biological sample (e.g., a tumor, blood or tissue sample) of a patient having a resistant and/or refractory Wnt-associated cancer ex vivo with a DNAPK inhibitor.

Wnt-associated cancers include, but are not limited to, solid tumors (such as gastric cancer, breast cancer, endometrial cancer, uterine cancer, colorectal cancer, synovial sarcoma, pancreatic cancer, melanoma, lobular carcinoma, prostate cancer, triple negative breast cancer (TNBC), non-small cell lung cancer (NSCLC), squamous cell lung carcinoma, lung adenocarcinoma, hepatocellular cancer (HCC), ovarian cancer, adenoid carcinoma, adrenocortical carcinoma, bladder/urothelial carcinoma, glioblastoma multiforme (GBM), cervical cancer, head and neck squamous cell carcinoma (HNSCC), kidney cancer, and thyroid cancer) and hematologic malignancies (such as acute myelogenous leukemia (AML), acute lymphoblastic leukemia (ALL), multiple myeloma (MM), chronic lymphocytic leukemia (CLL), chronic myelogenous leukemia (CML)), as well as cancer stem cells in many tumors types, particularly those described herein. In one embodiment, the Wnt-associated cancer is castration resistant prostate cancer.

Further provided herein are methods for treating or preventing androgen deprivation therapy-resistant cancers, comprising administering an effective amount of a DNAPK inhibitor to a patient having an androgen deprivation therapy-resistant cancer. In some embodiments, the method additionally comprises administering and effective amount of a Wnt pathway modulator, a Wnt inhibitor, and/or an androgen receptor (AR) antagonist.

Androgen deprivation therapy-resistant cancers include, but are not limited to, castration-resistant prostate cancer and AR positive tumors, such as breast cancer, cervical cancer, endometrial cancer, liver cancer, melanoma, ovarian cancer, renal cancer, skin cancer, testicular cancer, and urothelial cancer (http://www.proteinatlas.org/ENSG00000169083-AR/cancer). In one embodiment, the AR antagonist is Enzalutamide.

Further provided herein are methods for preventing androgen deprivation therapy resistance in cancers, comprising administering an effective amount of a DNAPK inhibitor to a patient having cancer. In some embodiment, the cancer is prostate cancer, breast cancer, cervical cancer, endometrial cancer, liver cancer, melanoma, ovarian cancer, renal cancer, skin cancer, testicular cancer, or urothelial cancer. In some embodiments, the method additionally comprises administering and effective amount of a Wnt pathway modulator as described herein and/or an androgen receptor (AR) antagonist.

Further provided herein are methods for treating or preventing enzalutamide-resistant cancers, comprising administering an effective amount of a DNAPK inhibitor to a patient having an enzalutamide-resistant cancer. In some embodiments, the method additionally comprises administering and effective amount of an androgen receptor (AR) antagonist.

Enzalutamide-resistant cancers include, but are not limited to, castration-resistant prostate cancer and AR positive tumors, such as breast cancer, cervical cancer, endometrial cancer, liver cancer, melanoma, ovarian cancer, renal cancer, skin cancer, testicular cancer, and urothelial cancer (http://www.proteinatlas.org/ENSG00000169083-AR/cancer).

Further provided herein are methods for detecting or measuring the inhibition of DNAPK activity in a patient, comprising measuring decreased phosphorylation of a DNAPK substrate (such as DNAPK or Hsp90a) in a biological sample from said patient, for example a peripheral blood or tumor sample, prior to and after the administration of a DNAPK inhibitor to said patient.

Further provided herein are methods for detecting or measuring the effect of inhibition of DNAPK activity on markers of Wnt activity in a patient, comprising measuring the markers of Wnt activity, as described herein, in a biological sample from said patient, for example a peripheral blood or tumor sample, prior to and after the administration of a DNAPK inhibitor to said patient, wherein modulation of markers of Wnt activity in a biological sample from said patient after administration of said DNAPK inhibitor relative to the markers of Wnt activity in a biological sample from said patient prior to administration of said DNAPK inhibitor indicates inhibition of DNAPK activity. In one embodiment, the marker of Wnt activity is one or more of CCND1, TCF7, Wnt1, FZD5, FZD1, TCF7L2, FZD6, AXIN1, FZD4, LEF1, CTBP1, LRP5, FZD8, WIF1, WNT7B, WNT3A, CD44, HNF4A, BTRC, LRP6, CTNNB1, WNT7A, WNT16, WNT8A, WNT3, WNT6, WNT4, WNT10A, CCND2, FZD9, AXIN2, TCF7L1, APC, cMYC, WNT2B, FZD3, or NFAT5. In another embodiment, the marker of Wnt activity is one or more of DNAPK, Axin2, FZD6, LEF1, FZD4, FZD8, CCND2, CCND1, cMYC, CTNNB1, Axin1, Wnt4, FZD9, Wnt16, Wnt6, LRP6, CTBP1, CD44, FZD3, Wnt2B, TCF7L2, Wnt7A, TCF7, Wnt2, Wnt3, Wnt3A, LRP5, APC, Wnt8A, or Wnt1. In another embodiment, the marker of Wnt activity is one or more of DNAPKFZD6, LRP5, LRP6, APC, FZD8, Wnt4, Wnt3A, BTRC, FZD3, CD44cMYC, Wnt10A, CTNNB1, CTBP1, Wnt2B, TCF7L2, FZD9, CCND1Axin1, Wnt3, FZD5, Axin2, Wnt1, TCF7L1, TCF7, LEF1, FZD1, Wnt8A, or CCND2.

Also provided herein are methods for predicting the likelihood of a cancer of a patient being responsive to DNAPK inhibitor therapy, comprising screening a biological sample of said patient for markers of Wnt activity, wherein the presence of markers of Wnt activity in a biological sample of said patient indicates an increased likelihood that a cancer of said patient will be responsive to DNAPK inhibitor therapy. In one embodiment, the marker of Wnt activity is one or more of CCND1, TCF7, Wnt1, FZD5, FZD1, TCF7L2, FZD6, AXIN1, FZD4, LEF1, CTBP1, LRP5, FZD8, WIF1, WNT7B, WNT3A, CD44, HNF4A, BTRC, LRP6, CTNNB1, WNT7A, WNT16, WNT8A, WNT3, WNT6, WNT4, WNT10A, CCND2, FZD9, AXIN2, TCF7L1, APC, cMYC, WNT2B, FZD3, or NFAT5. In another embodiment, the marker of Wnt activity is one or more of DNAPK, Axin2, FZD6, LEF1, FZD4, FZD8, CCND2, CCND1, cMYC, CTNNB1, Axin1, Wnt4, FZD9, Wnt16, Wnt6, LRP6, CTBP1, CD44, FZD3, Wnt2B, TCF7L2, Wnt7A, TCF7, Wnt2, Wnt3, Wnt3A, LRP5, APC, Wnt8A, or Wnt1. In another embodiment, the marker of Wnt activity is one or more of DNAPK, FZD6, LRP5, LRP6, APC, FZD8, Wnt4, Wnt3A, BTRC, FZD3, CD44cMYC, Wnt10A, CTNNB1, CTBP1, Wnt2B, TCF7L2, FZD9, CCND1Axin1, Wnt3, FZD5, Axin2, Wnt1, TCF7L1, TCF7, LEF1, FZD1, Wnt8A, or CCND2.

Further provided herein are methods for determining whether a patient is sensitive to a DNAPK inhibitor, comprising administering to said patient said DNAPK inhibitor and determining whether markers of Wnt activity are modulated in said patient by measuring the markers of Wnt activity in a biological sample from said patient, for example a peripheral blood or tumor sample, prior to and after the administration of the DNAPK inhibitor to said patient, wherein changes in markers of Wnt activity by said DNAPK inhibitor indicates that a patient is sensitive to said DNAPK inhibitor. In one embodiment, the marker of Wnt activity is one or more of CCND1, TCF7, Wnt1, FZD5, FZD1, TCF7L2, FZD6, AXIN1, FZD4, LEF1, CTBP1, LRP5, FZD8, WIF1, WNT7B, WNT3A, CD44, HNF4A, BTRC, LRP6, CTNNB1, WNT7A, WNT16, WNT8A, WNT3, WNT6, WNT4, WNT10A, CCND2, FZD9, AXIN2, TCF7L1, APC, cMYC, WNT2B, FZD3, or NFAT5. In another embodiment, the marker of Wnt activity is one or more of DNAPK, Axin2, FZD6, LEF1, FZD4, FZD8, CCND2, CCND1, cMYC, CTNNB1, Axin1, Wnt4, FZD9, Wnt16, Wnt6, LRP6, CTBP1, CD44, FZD3, Wnt2B, TCF7L2, Wnt7A, TCF7, Wnt2, Wnt3, Wnt3A, LRP5, APC, Wnt8A, or Wnt1. In another embodiment, the marker of Wnt activity is one or more of DNAPK, FZD6, LRP5, LRP6, APC, FZD8, Wnt4, Wnt3A, BTRC, FZD3, CD44cMYC, Wnt10A, CTNNB1, CTBP1, Wnt2B, TCF7L2, FZD9, CCND1Axin1, Wnt3, FZD5, Axin2, Wnt1, TCF7L1, TCF7, LEF1, FZD1, Wnt8A, or CCND2.

Also provided herein is a kit for detecting markers of Wnt activity in a biological sample from a patient before and after treatment with a DNAPK inhibitor, comprising reagents for measuring markers of Wnt activity and one or more DNAPK markers. In one embodiment, the marker of Wnt activity is one or more of CCND1, TCF7, Wnt1, FZD5, FZD1, TCF7L2, FZD6, AXIN1, FZD4, LEF1, CTBP1, LRP5, FZD8, WIF1, WNT7B, WNT3A, CD44, HNF4A, BTRC, LRP6, CTNNB1, WNT7A, WNT16, WNT8A, WNT3, WNT6, WNT4, WNT10A, CCND2, FZD9, AXIN2, TCF7L1, APC, cMYC, WNT2B, FZD3, or NFAT5. In another embodiment, the marker of Wnt activity is one or more of DNAPK, Axin2, FZD6, LEF1, FZD4, FZD8, CCND2, CCND1, cMYC, CTNNB1, Axin1, Wnt4, FZD9, Wnt16, Wnt6, LRP6, CTBP1, CD44, FZD3, Wnt2B, TCF7L2, Wnt7A, TCF7, Wnt2, Wnt3, Wnt3A, LRP5, APC, Wnt8A, or Wnt1. In another embodiment, the marker of Wnt activity is one or more of DNAPK. FZD6, LRP5, LRP6, APC, FZD8, Wnt4, Wnt3A, BTRC, FZD3, CD44cMYC, Wnt10A, CTNNB1, CTBP1, Wnt2B, TCF7L2, FZD9, CCND1Axin1, Wnt3, FZD5, Axin2, Wnt1, TCF7L1, TCF7, LEF1, FZD1, Wnt8A, or CCND2.

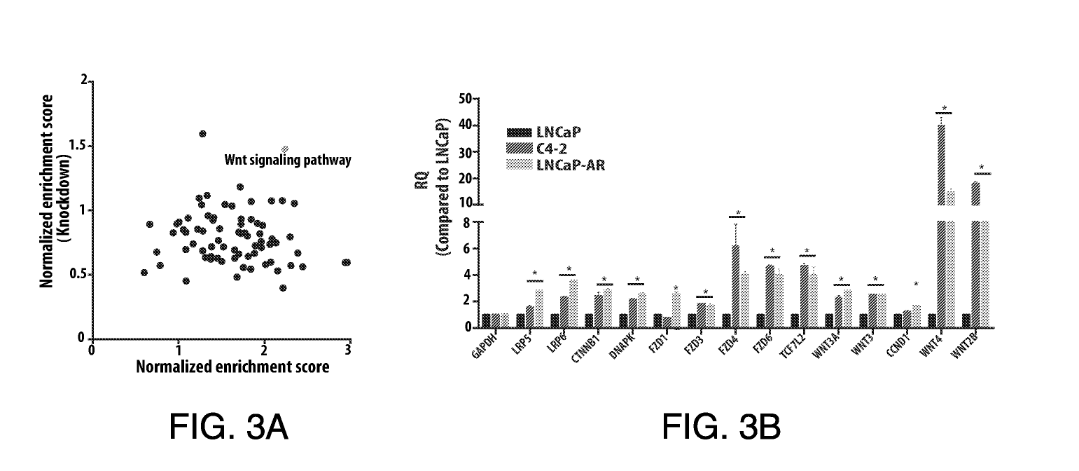

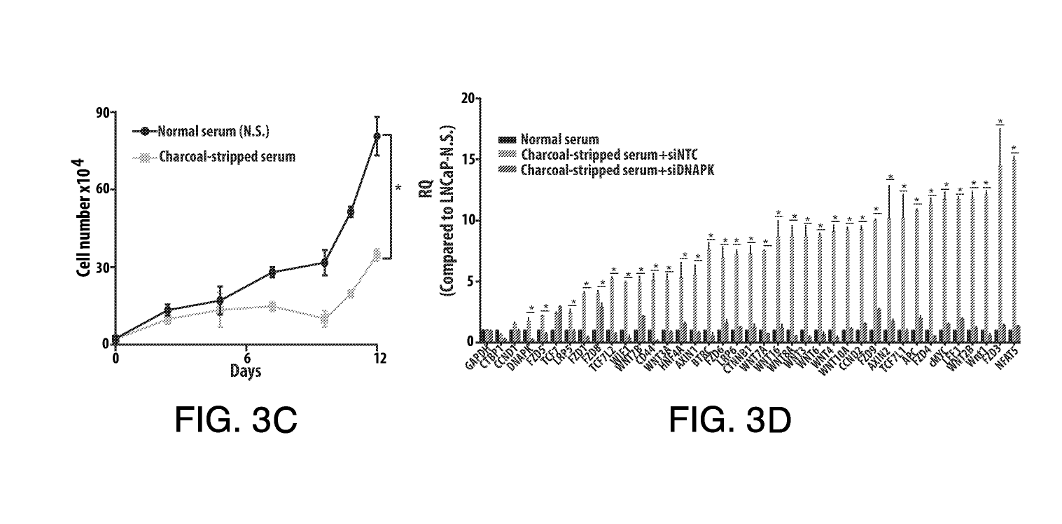

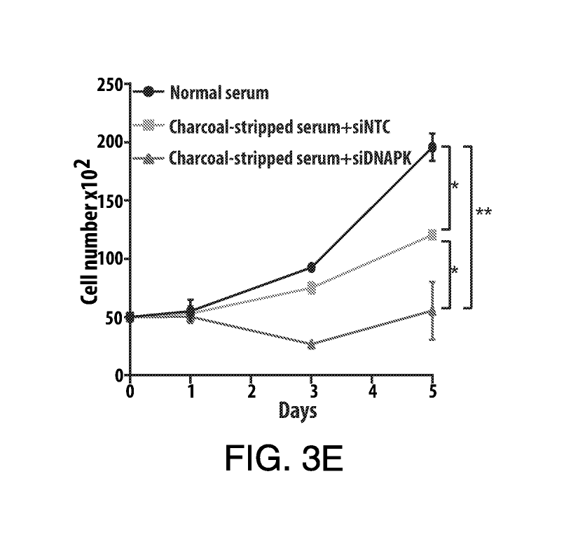

Methods for identifying Wnt-associated cancers are known in the art (see, e.g., Tumova, L et al. Mol Cancer Ther April 2014 13:812-822; Takebe et al. Nat Rev Clin Oncol. 2015 Apr. 7. doi: 10.1038/nrclinonc.2015.61. (Epub ahead of print); Madan N S and Virshup, Mol Cancer Ther. 2015 May; 14(5):1087-1094. Chiurillo, Mass. World J Exp Med. 2015 May 20; 5(2):84-102; Ashihara, E et al. Cancer Sci. 2015 June; 106(6):665-71. Illustrative methods to evaluate markers of Wnt activity (mutations, copy number variations (CNV's, gains or losses), fusions, decreased/increased expression (see FIG. 3) or mislocalization of miRNA, mRNA or protein, or changes in phosphorylation or activity of Wnt pathway genes or regulators) include, but are not limited to sequencing, PCR, FISH, microarrays, RT-PCR, immunohistochemistry (IHC), western blots, ELISAs.

In some embodiments, the DNAPK inhibitor is a compound as described herein. In one embodiment, the DNAPK inhibitor is Compound 1 (a DNAPK inhibitor set forth herein having molecular formula C₁₆H₁₆N₈O). In one embodiment, Compound 1 is 1-ethyl-7-(2-methyl-6-(1H-1,2,4-triazol-3-yl)pyridin-3-yl)-3,4-dihydropyrazino[2,3-b]pyrazin-2(1H)-one, or a tautomer thereof, for example, 1-ethyl-7-(2-methyl-6-(4H-1,2,4-triazol-3-yl)pyridin-3-yl)-3,4-dihydropyrazino[2,3-b]pyrazin-2(1H)-one, or 1-ethyl-7-(2-methyl-6-(1H-1,2,4-triazol-5-yl)pyridin-3-yl)-3,4-dihydropyrazino[2,3-b]pyrazin-2(1H)-one.

A DNAPK inhibitor can be combined with radiation therapy or surgery. In certain embodiments, a DNAPK inhibitor is administered to patient who is undergoing radiation therapy, has previously undergone radiation therapy or will be undergoing radiation therapy. In certain embodiments, a DNAPK inhibitor is administered to a patient who has undergone tumor removal surgery. In some embodiments of the methods described herein, the methods additionally comprise administration of a Wnt pathway modulator, a Wnt inhibitor and/or an androgen receptor antagonist, as described herein. In certain embodiments, a DNAPK inhibitor can be administered before, after or simultaneously with a Wnt pathway modulator or a Wnt inhibitor in the methods provided herein. A DNAPK inhibitor can also be combined with an AR antagonist such as enzalutamide in the methods provided herein. In certain embodiments, a DNAPK inhibitor can be administered before, after or simultaneously with an AR antagonist such as enzalutamide in the methods provided herein.

### 4.6 Pharmaceutical Compositions and Routes of Administration

Provided herein are compositions, comprising an effective amount of a DNAPK inhibitor, and compositions comprising an effective amount of a DNAPK inhibitor and a pharmaceutically acceptable carrier or vehicle. In some embodiments, the pharmaceutical compositions described herein are suitable for oral, parenteral, mucosal, transdermal or topical administration.

The DNAPK inhibitors can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the DNAPK inhibitor in the pharmaceutical composition may be at a level that will exercise the desired effect; for example, about 0.005 mg/kg of a patient's body weight to about 10 mg/kg of a patient's body weight in unit dosage for both oral and parenteral administration.

The dose of a DNAPK inhibitor to be administered to a patient is rather widely variable and can be subject to the judgment of a health-care practitioner. In general, the DNAPK inhibitors can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight, about 0.25 mg/kg of a patient's body weight to about 0.5 mg/kg of a patient's body weight, or about 0.007 mg/kg of a patient's body weight to about 1.7 mg/kg of patient's body weight. In one embodiment, one dose is given per day. In another embodiment, two doses are given per day. In any given case, the amount of the DNAPK inhibitor administered will depend on such factors as the solubility of the active component, the formulation used and the route of administration.

In another embodiment, provided herein are methods for the treatment or prevention of a DNAPK and Wnt-associated cancer, comprising the administration of about 0.375 mg/day to about 750 mg/day, about 0.75 mg/day to about 375 mg/day, about 3.75 mg/day to about 75 mg/day, about 7.5 mg/day to about 55 mg/day, about 18 mg/day to about 37 mg/day, about 0.5 mg/day to about 60 mg/day, or about 0.5 mg/day to about 128 mg/day of a DNAPK inhibitor to a patient in need thereof. In another embodiment, provided herein are methods for the treatment or prevention of a DNAPK and Wnt-associated cancer, comprising the administration of about 0.5 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of a DNAPK inhibitor to a patient in need thereof. In a particular embodiment, the methods disclosed herein comprise the administration of 0.5 mg/day, 1 mg/day, 2 mg/day, 4 mg/day, 8 mg/day, 16 mg/day, 20 mg/day, 25 mg/day, 30 mg/day, 45 mg/day, 60 mg/day, 90 mg/day, 120 mg/day or 128 mg/day of a DNAPK inhibitor to a patient in need thereof.

In another embodiment, provided herein are unit dosage formulations that comprise between about 0.1 mg and about 2000 mg, about 1 mg and 200 mg, about 35 mg and about 1400 mg, about 125 mg and about 1000 mg, about 250 mg and about 1000 mg, or about 500 mg and about 1000 mg of a DNAPK inhibitor.

In a particular embodiment, provided herein are unit dosage formulation comprising about 0.1 mg, 0.25 mg, 0.5 mg, 1 mg, 5 mg, 7.5 mg, 10 mg, 15 mg, 20 mg, 30 mg, 45 mg, 50 mg, 60 mg, 75 mg, 100 mg, 125 mg, 150 mg, 200 mg, 250 mg, 300 mg, 400 mg, 600 mg or 800 mg of a DNAPK inhibitor.

In another embodiment, provided herein are unit dosage formulations that comprise 0.1 mg, 0.25 mg, 0.5 mg, 1 mg, 2.5 mg, 5 mg, 7.5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a DNAPK inhibitor. In a particular embodiment, provided herein are unit dosage formulations that comprise 5 mg, 7.5 mg, 10 mg, 15 mg, 20 mg, 30 mg, 45 mg or 60 mg of a DNAPK inhibitor.

A DNAPK inhibitor can be administered once, twice, three, four or more times daily.

A DNAPK inhibitor can be administered orally for reasons of convenience. In one embodiment, when administered orally, a DNAPK inhibitor is administered with a meal and water. In another embodiment, the DNAPK inhibitor is dispersed in water or juice (e.g., apple juice or orange juice) and administered orally as a suspension. In another embodiment, when administered orally, a DNAPK inhibitor is administered in a fasted state.

The DNAPK inhibitor can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a DNAPK inhibitor without an additional carrier, excipient or vehicle.

In another embodiment, provided herein are compositions, comprising an effective amount of a DNAPK inhibitor and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a DNAPK inhibitor with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a DNAPK inhibitor as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the DNAPK inhibitor can be delayed or prolonged by proper formulation. For example, a slowly soluble pellet of the DNAPK inhibitor can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the DNAPK inhibitor in oily or emulsified vehicles that allow it to disperse slowly in the serum.

### 4.7 Kits

In certain embodiments, provided herein are kits comprising a DNAPK inhibitor.

In other embodiments, provide herein are kits comprising a DNAPK inhibitor and means for monitoring patient response to administration of said DNAPK inhibitor. In certain embodiments, the patient has a Wnt-associated cancer. In particular embodiments, the patient response measured is inhibition of disease progression, inhibition of tumor growth, reduction of primary and/or secondary tumor(s), relief of tumor-related symptoms, improvement in quality of life, delayed appearance of primary and/or secondary tumors, slowed development of primary and/or secondary tumors, decreased occurrence of primary and/or secondary tumors, slowed or decreased severity of secondary effects of disease, arrested tumor growth or regression of tumor.

In other embodiments, provided herein are kits comprising a DNAPK inhibitor and means for measuring markers of Wnt activity in a patient. In certain embodiments, the kits comprise means for measuring markers of Wnt activity in circulating blood or tumor cells and/or skin biopsies or tumor biopsies/aspirates of a patient. In certain embodiments, provided herein are kits comprising a DNAPK inhibitor and means for measuring markers of Wnt activity as assessed by comparison of the markers of Wnt activity before, during and/or after administration of the DNAPK inhibitor. In certain embodiments, provided herein are kits comprising a DNAPK inhibitor and means for measuring markers of DNAPK activity as assessed by comparison of the markers of DNAPK activity before, during and/or after administration of the DNAPK inhibitor.

In certain embodiments, the kits provided herein further comprise instructions for use, such as for administering a DNAPK inhibitor and/or monitoring patient response to administration of a DNAPK inhibitor.

### 5. EXAMPLES

### 5.1 Biological Examples

### 5.1.1 DNAPK is the Top Differentially Expressed Kinase Associated with Metastatic Progression of CRPC

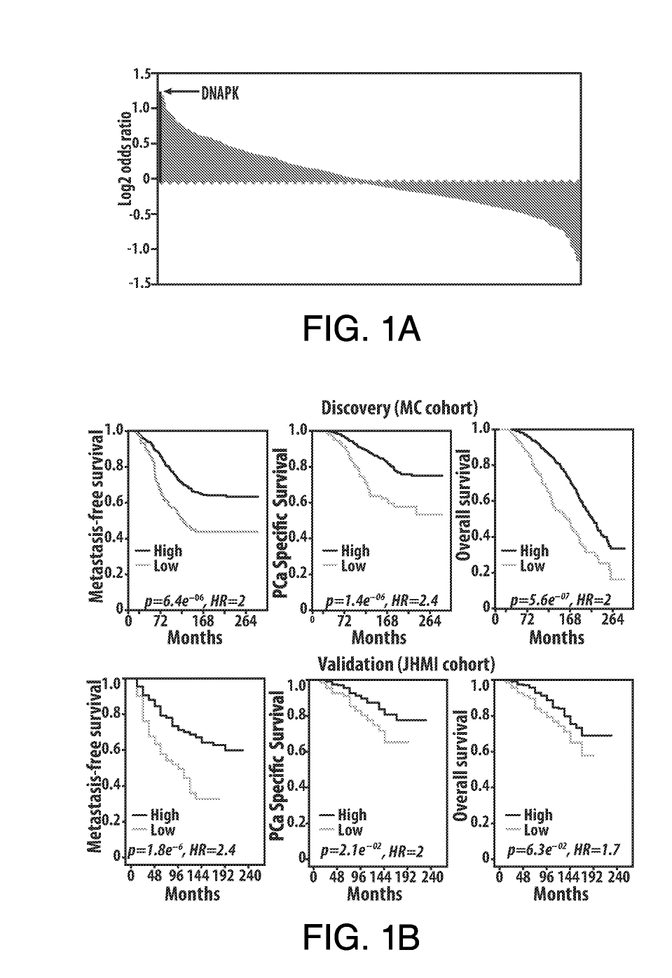

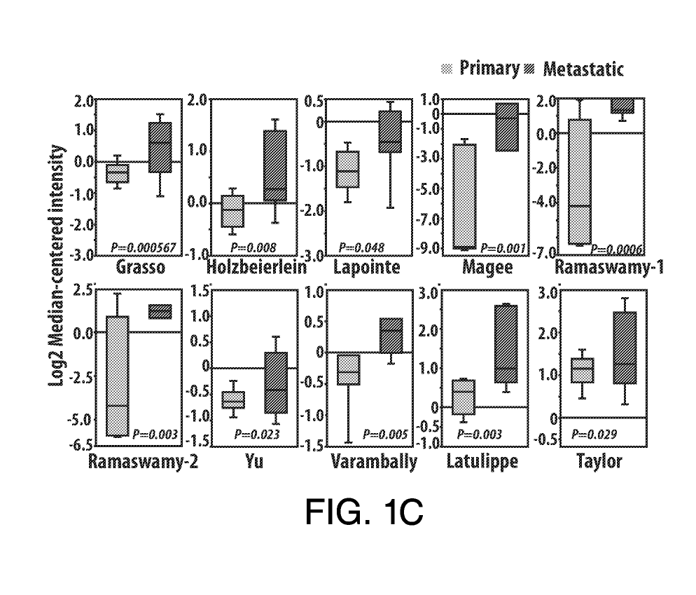

Association of expression of all known kinases with metastatic progression in prostate cancer samples was examined in a cohort of patients treated with prostatectomy. Notable features of this cohort include the long clinical follow-up (median of 13.4 years), the large sample size (n=545), and the prevalence of high-risk characteristics as defined by National Comprehensive Cancer Network criteria (www.nccn.org), such as extracapsular extension (50%) and seminal vesicle invasion (32%). Consistent with these features, 39% of the patients experienced metastatic progression. In this discovery cohort, kinases were ranked by the relative enrichment for metastatic progression in cases with high versus low expression of each kinase, with expression cut-offs defined by an unbiased clustering approach, as described in methods. This analysis demonstrated that DNAPK was the top kinase that enriched for metastatic progression (OR=2.19, p<0.0001, FIG. 1 (panel A)). The prognostic impact of elevated DNAPK expression in prostate cancer patients in this cohort showed that high DNAPK was significantly associated with not only metastatic progression (HR=2.0 [1.5-2.7], p<0.0001), but also decreased rates of prostate cancer-specific survival (HR=2.4 [1.7-3.5], p<0.0001) and overall survival (HR=2.0 [1.5-2.6], p<0.0001) (FIG. 1 (panel B)). These findings were validated in an independent cohort of patients where high DNAPK expression again associated with increased metastatic progression (HR=2.4 [1.7-3.6], p<0.0001) and decreased prostate cancer-specific survival (HR=2 [1.1-3.5], p<0.02), with borderline significance for decreased overall survival (HR=1.7 [0.97-2.8], p<0.06) (FIG. 1 (panel B)). After identifying the prognostic value of DNAPK in localized prostate cancer, expression of DNAPK was examined in metastatic prostate cancer using Oncomine analysis (www.oncomine.org). DNAPK was significantly overexpressed in metastatic versus primary tumor prostate cancer samples in 10/12 cohorts (FIG. 1 (panel C)). These results show that DNAPK is strongly prognostic in localized high-risk prostate cancer, and associated with the development of metastatic disease.

### 5.1.2 Effect of Knock Down or Inhibition of DNAPK on Aggressive Cancer Phenotypes In Vitro

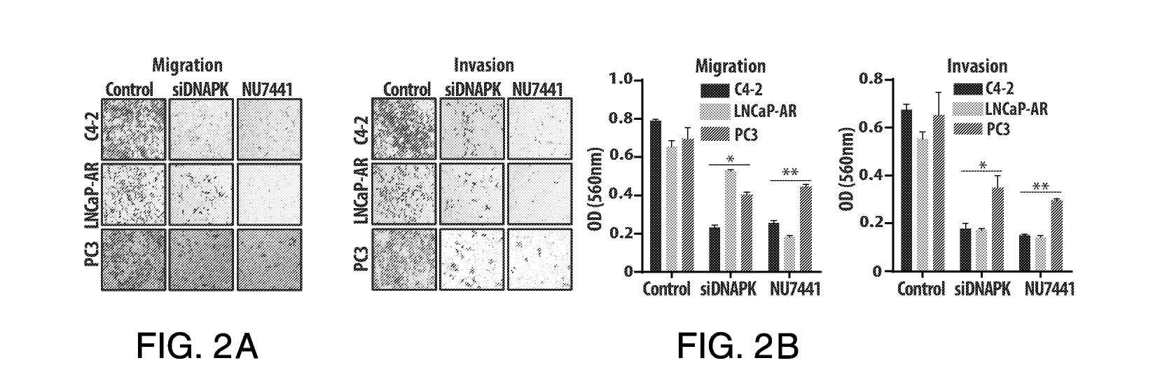

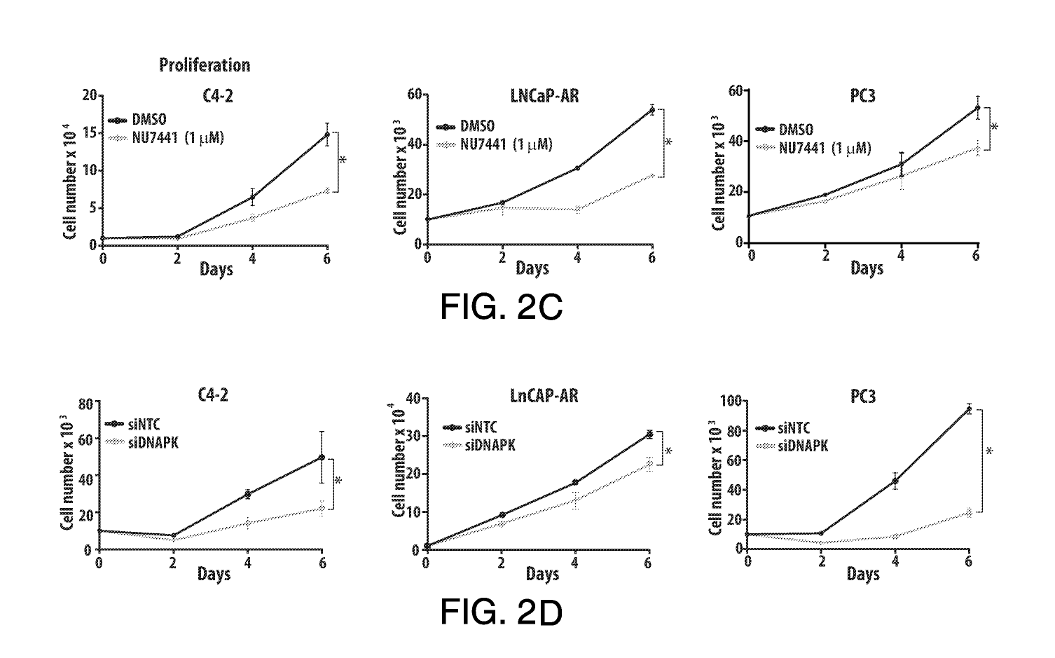

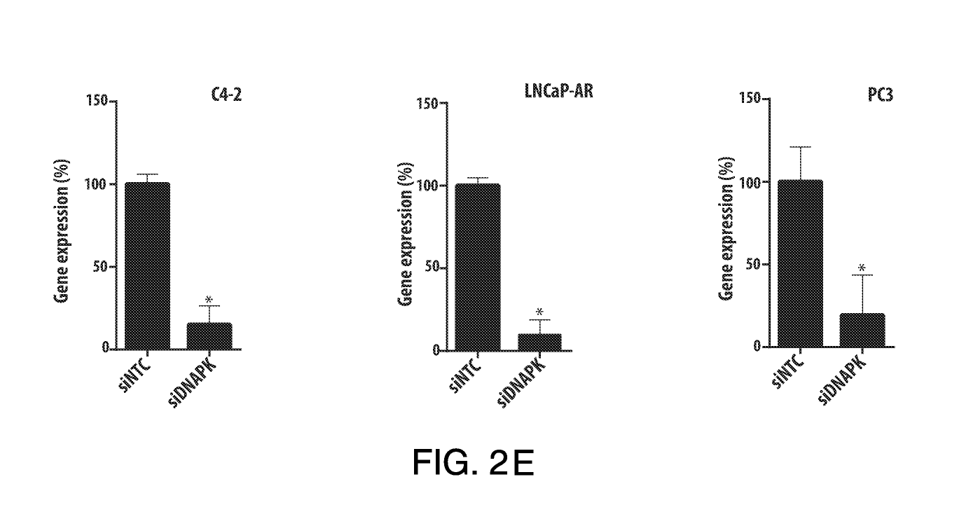

Knockdown (via siRNAs) or pharmacological inhibition (via the drug NU7441) of DNAPK drastically diminished the migration, invasion and proliferation of both AR-positive cells LNCaP-AR and C4-2, as well as AR-negative PC3 cells (FIG. 2 (panels A-D)). Efficient knockdown of DNAPK was achieved in these experiments (FIG. 2 (panel E)). The reduction in aggressive cancer phenotypes in AR-negative PC3 cells indicated DNAPK has functions beyond regulating AR activity that may contribute to oncogenic phenotypes in prostate cancer.

### 5.1.3 Effect of DNAPK on Wnt Signaling Pathway in Promotion of Aggressive Prostate Cancer

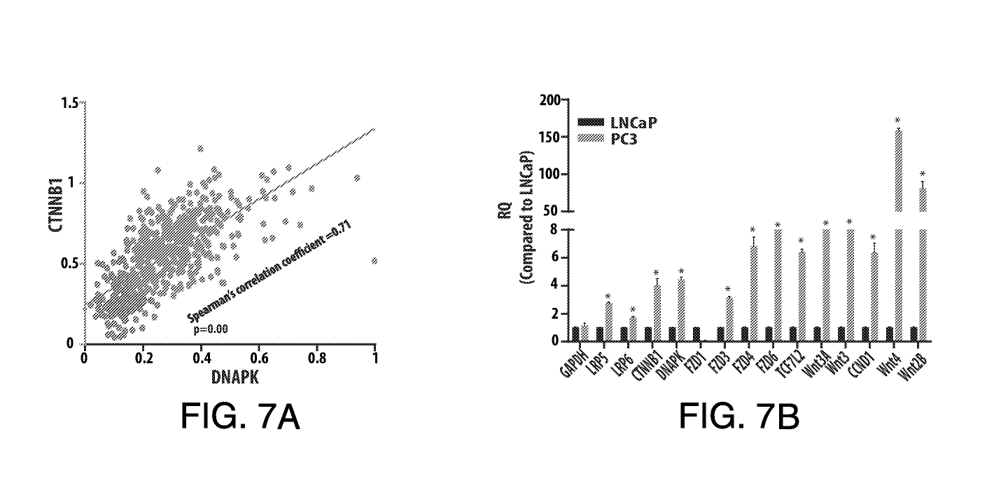

A list of genes that were significantly changed after DNAPK knockdown in VCaP, C4-2B, PC3 and DU145 cells by microarray and a list of genes correlated with the DNAPK expression were generated based on guilt-by-association analyses in vitro and in vivo. GSEA of these gene lists produced normalized enrichment scores (NES) for pathway gene sets. A scatterplot of gene set pathways was generated with in vitro NES value on the y-axis, and with in vivo NES value on the x-axis (FIG. 3 (panel A)) as well as the greatest area under the curve (AUC=3.642). The Wnt pathway had the top average NES (knockdown NES=1.48, NES=2.47, average=1.97, FIG. 3 (panel A)), and the most area under the curve (AUC=3.642). It was found that the expression of DNAPK was tightly correlated with the expression of beta catenin (Spearman's correlation coefficient=0.71, FIG. 7 (panel A). These data demonstrated that Wnt signaling as a major pathway modulated by DNAPK, which is particularly intriguing based on the established deregulation and oncogenic role of Wnt signaling in metastatic prostate cancer.

Higher expression of Wnt pathway genes across the cell line models of disease progression was found, with LNCaP cells representing hormone-sensitive disease (LNCaP-AR), C4-2B cells representing castration-resistant prostate cancer (FIG. 3 (panel B)), and AR-independent PC3 cells representing neuro-endocrine component of prostate cancer (FIG. 7 (panel B)). Consistent with the clinical observations that hormone naive cells eventually overcome the ADT, it was found that LNCaP cells continued to grow despite hormone-depletion, albeit with a slower rate (FIG. 3 (panel C)). It was also found that the hormone-depletion triggered the expression of multiple Wnt genes in LNCaP cells, compared to LNCaP cells grown under normal serum conditions (FIG. 3 (panel D)). These data demonstrated that LNCaP cells are a good model of Wnt signaling induction upon androgen deprivation.

### 5.1.4 Effect of Knock Down of DNAPK on Androgen-Depletion-Induced Wnt Signaling

It was observed that siRNA-mediated knockdown of DNAPK in LNCaP cells grown under hormone-depletion conditions abrogated the expression of Wnt genes (FIG. 3 (panel E)), indicating that DNAPK is required for ADT-induced Wnt signaling.

### 5.1.5 Effect of DNAPK Inhibition on Wnt-Induced Cancer Phenotypes in CRPC Cells

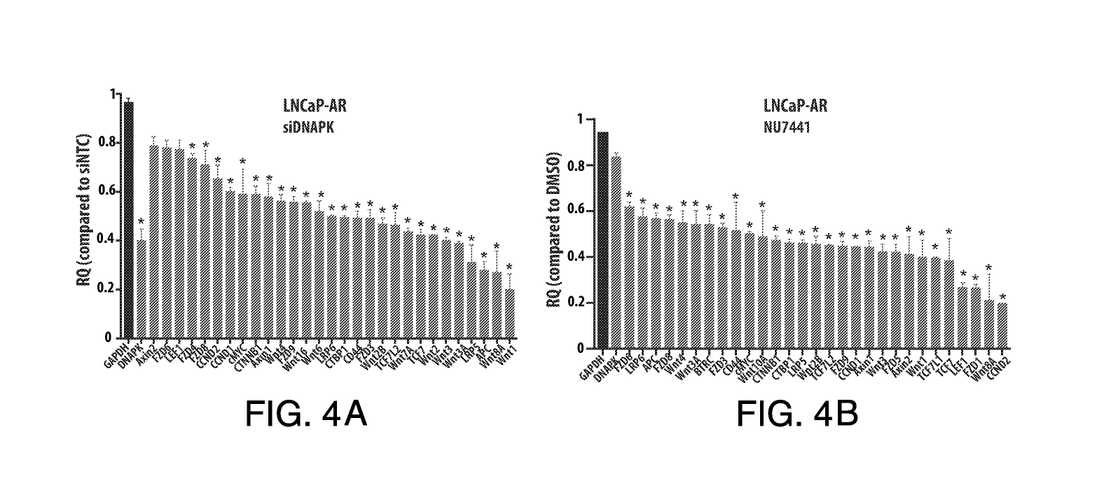

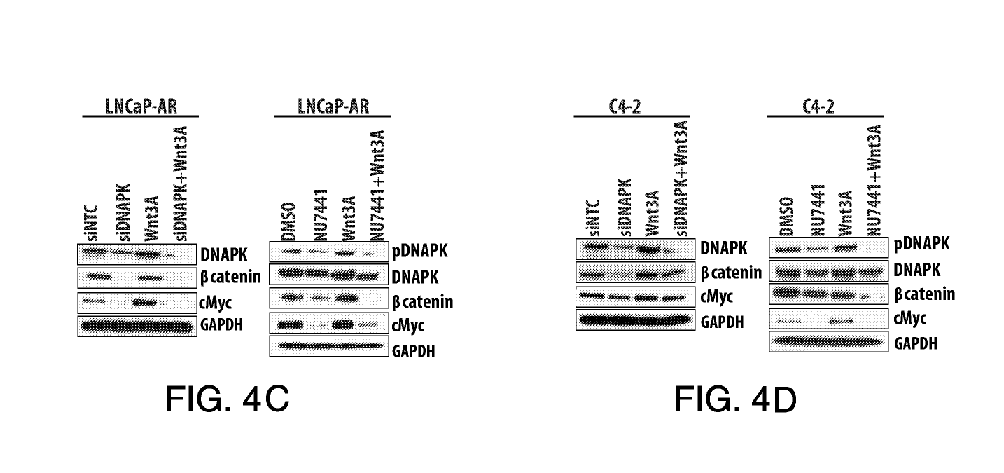

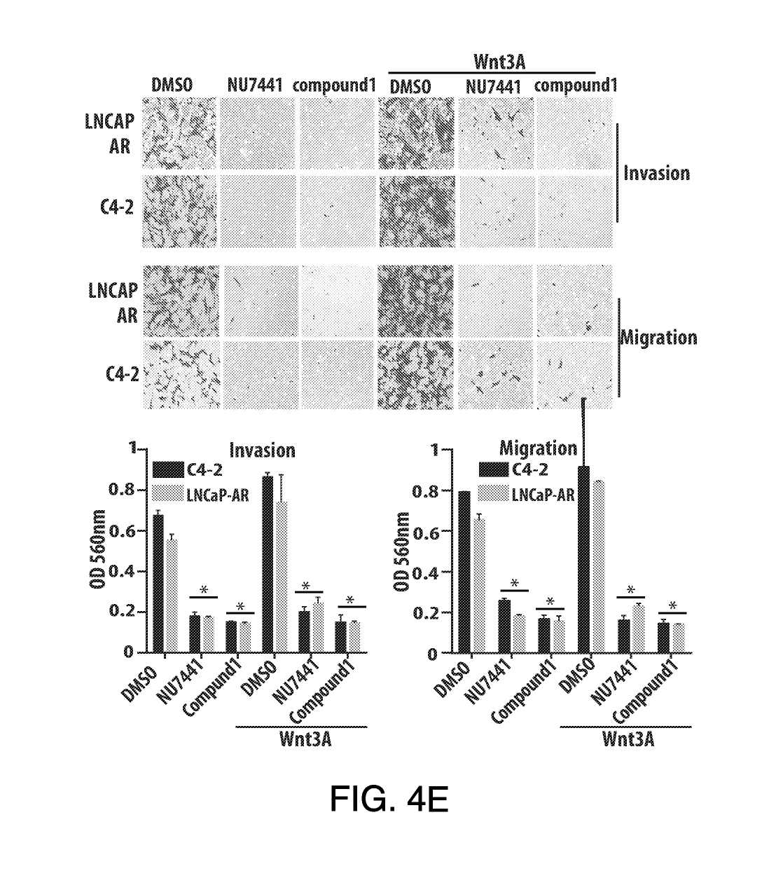

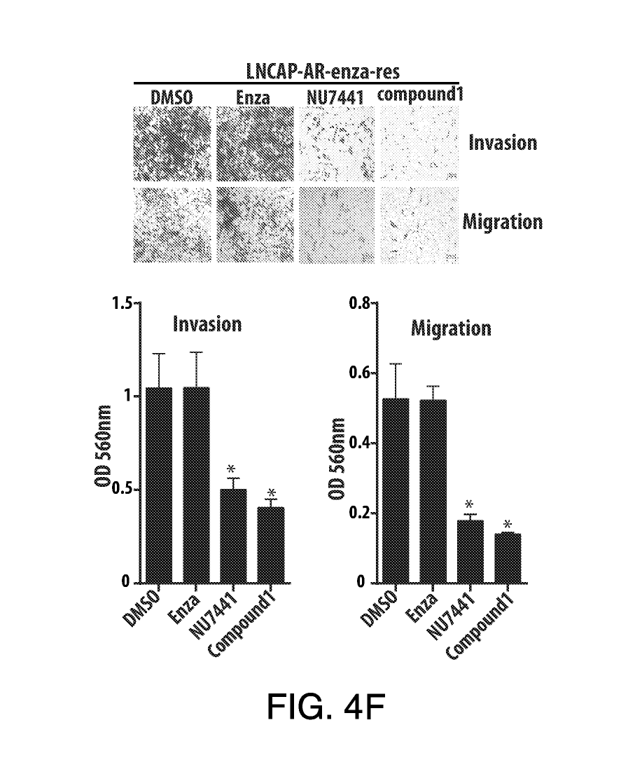

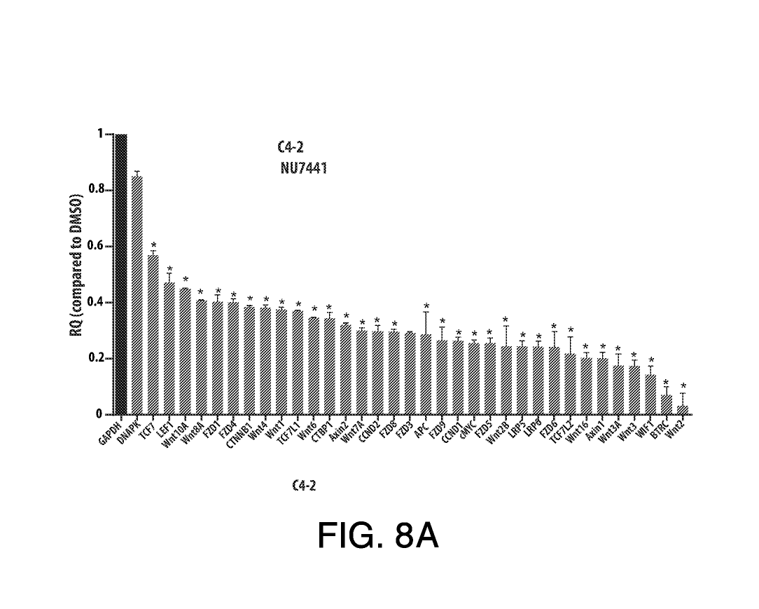

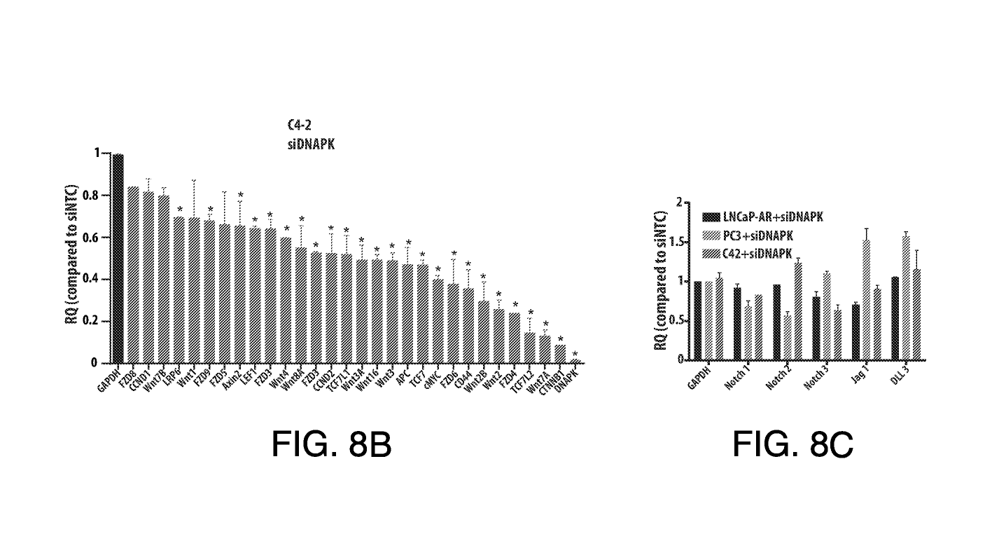

A robust reduction of Wnt pathway genes after DNAPK silencing or inhibition in CRPC cells (LNCaP-AR and C4-2) (FIG. 4 (panels A-B) and FIG. 8 (panels A-B)) was found, while genes implicated in other pathway (i.e. notch) remain unchanged (FIG. 8 (panel C)). It was also found that DNAPK knockdown or inhibition with NU7441 reduced baseline and Wnt3A-induced levels of active beta-catenin and cMyc (a classical target of Wnt signaling) (FIG. 4 (panels C-D)). Phenotypically, DNAPK inhibition with NU7441 or Compound 1 abrogated baseline and Wnt3A-induced invasion and migration of CRPC cells (FIG. 4 (panel E)). These data indicate that Wnt signaling can drive oncogenic phenotypes in CRPC cells which can be blocked by DNAPK inhibition.

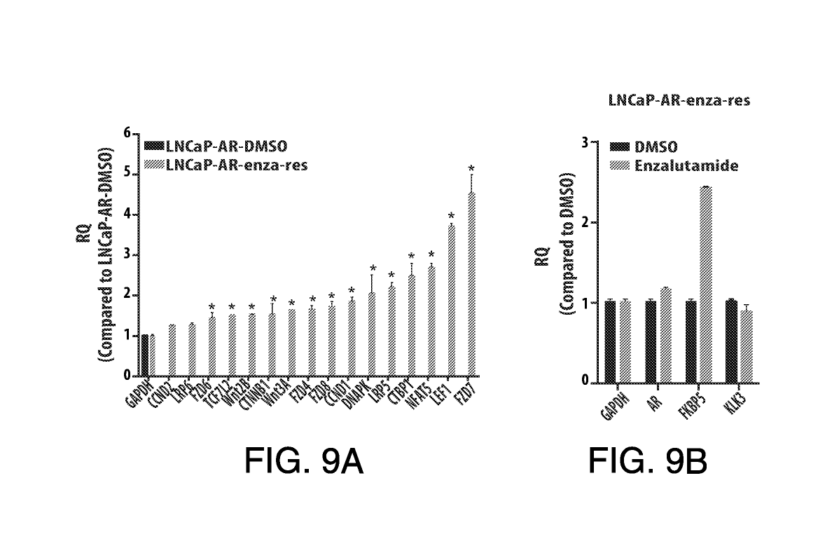

Increased expression of Wnt pathway genes in LNCaP-AR cells treated with low-dose enzalutamide until resistance emerged (LNCaP-AR-MDVR; FIG. 9 (panels A-B)) was also found. DNAPK inhibition with NU7441 or Compound 1 significantly reduced invasion and migration of LNCaP-AR-MDVR cells (FIG. 4 (panel F)). Thus, DNAPK inhibition is a potential therapeutic strategy in enzalutamide-resistant castration-resistant prostate cancer. Taken together, these data indicate that DNAPK inhibition is a highly promising therapeutic strategy at critical points in prostate cancer: at ADT initiation to prevent emergence of resistance, in ADT-resistant castration-resistant prostate cancer, and after emergence of enzalutamide resistance.

### 5.1.6 Wnt Signaling is a Major Target of DNAPK Inhibition

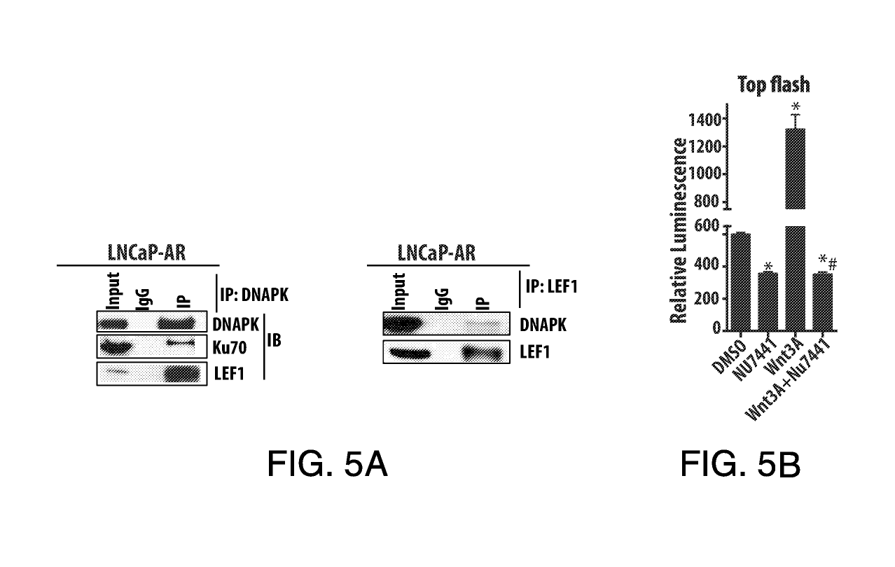

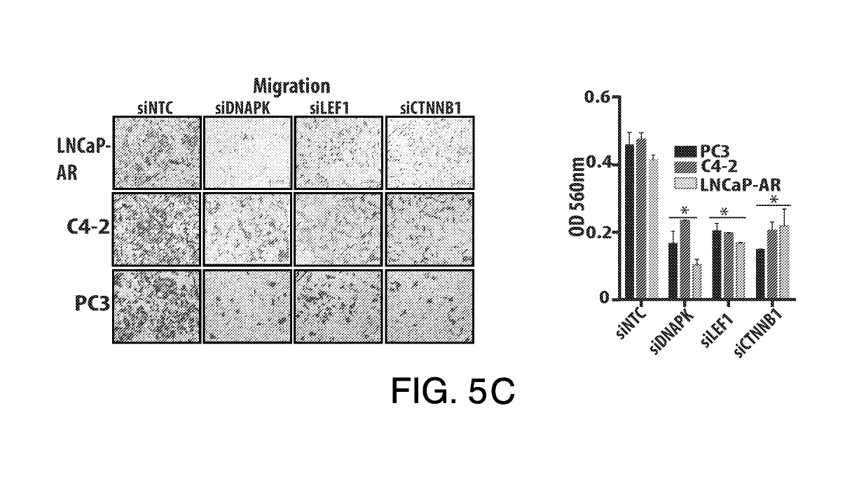

Co-immunoprecipitation studies revealed that DNAPK interacts with LEF1 (FIG. 5 (panel A)), a major transcription factor of the Wnt pathway. DNAPK inhibition with NU7441 significantly reduced baseline and Wnt3A-induced TOPFLASH reporter activity in PC3 cells (FIG. 5 (panel B)), indicating that DNAPK facilitates Wnt signaling through interaction with the Wnt pathway transcription factor LEF1. Downregulation of Wnt signaling with siRNAs targeting LEF1 or beta-catenin (CTNNB1) resulted in substantial reduction in invasion and migration of LNCaP, C4-2, and PC3 cells (FIG. 5 (panel C)), almost comparable to reductions achieved with DNAPK inhibition. These findings suggest that Wnt signaling blockade is a major component of the effects of DNAPK inhibition.

### 5.1.7 Effect of DNAPK Inhibition on CRPC Xenograft Growth In Vivo

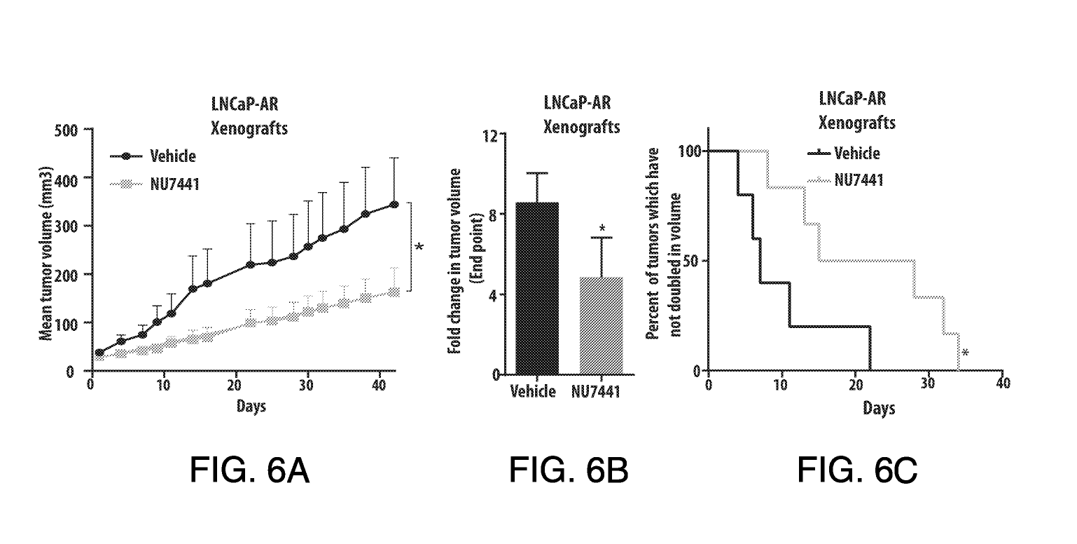

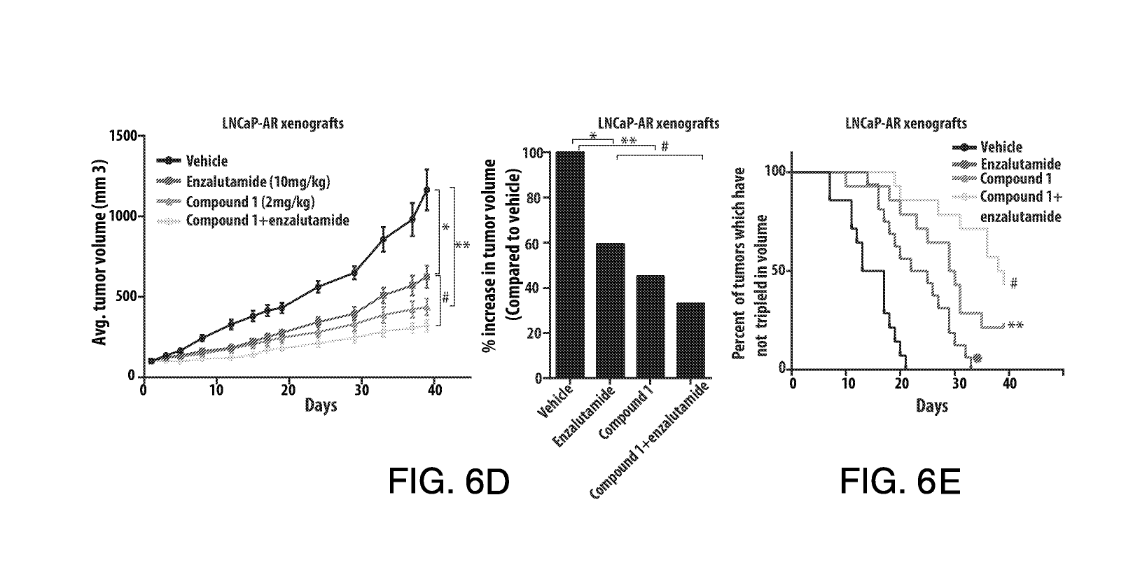

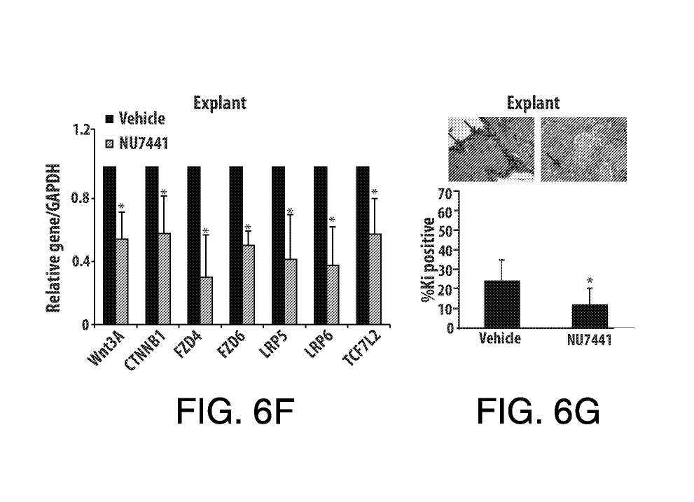

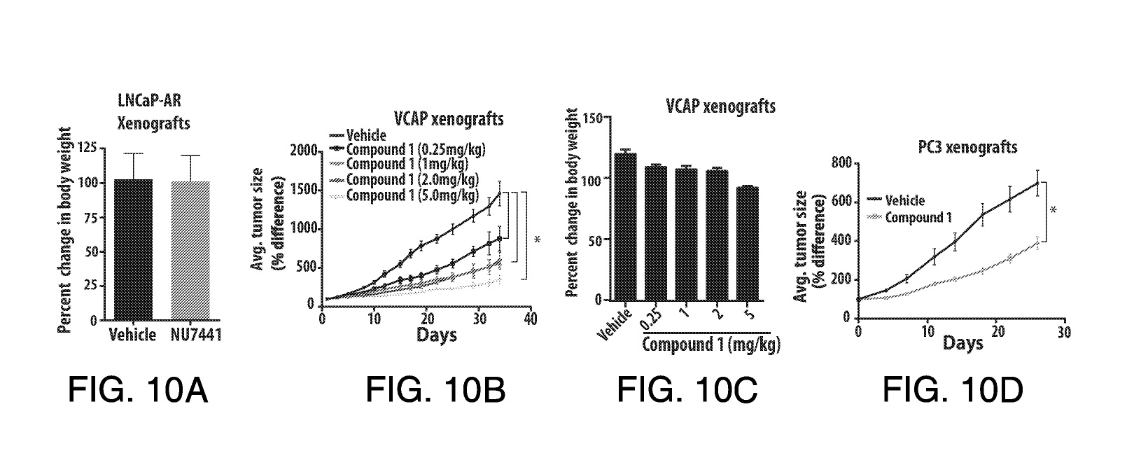

Palpable LNCaP-AR tumors in castrated mice were treated with DNAPK inhibitor NU7441. There was a 44% reduction in tumor growth with NU7441 treatment compared to control (relative tumor volume 4.84±1.97 with NU7441 vs 8.57±1.45 with vehicle, FIG. 6 (panels A-B)). This growth inhibition translated into a significant delay in tumor doubling time in the NU7441 arm (median 21.5 days with NU7441 vs 7 days with vehicle, FIG. 6 (panel C)). The toxicity was minimal as measured by body weight (FIG. 10 (panel A)). Using a VCaP xenograft model, it was established that Compound 1 induced a dose-dependent reduction in tumor growth, best observed at a non-toxic dose of 2 mg/kg (FIG. 10 (panels B-C). The effect of the same dose of Compound 1 on CRPC xenograft growth under pre-castrated conditions resulted in a significantly better tumor regression compared to enzalutamide (FIG. 6 (panel D)). A combination of Compound 1 and enzalutamide resulted in a significant delay in tumor growth compared to either monotherapies. This reduction was associated with significant delay in tumor tripling time in the Compound 1+enzalutamide arm, with median tumor tripling time of 38.5 days as compared to vehicle (15 days), Compound 1 (29.5 days) or enzalutamide (23.5 days) (FIG. 6 (panel E)). DNAPK inhibition via Compound 1 also resulted in a significant regression of AR-independent PC3 tumors (FIG. 10 (panel D)), confirming the potential of treatment strategies based on DNAPK inhibition in AR-independent disease. Consistent with the xenograft studies, human prostate cancer explants treated with NU7441 showed marked reduction in Wnt target gene expression (FIG. 6 (panel F)) and proliferative index, as determined by Ki67 levels (FIG. 6 (panel G)). Taken together, our results highlight the promise of DNAPK inhibition in the treatment of aggressive prostate cancer.

A number of references have been cited, the disclosures of which are incorporated herein by reference in their entirety. The embodiments disclosed herein are not to be limited in scope by the specific embodiments disclosed in the examples which are intended as illustrations of a few aspects of the disclosed embodiments and any embodiments that are functionally equivalent are encompassed by the present disclosure. Indeed, various modifications of the embodiments disclosed herein are in addition to those shown and described herein will become apparent to those skilled in the art and are intended to fall within the scope of the appended claims.

1. A method for treating or preventing a Wnt-associated cancer, comprising administering an effective amount of a DNAPK inhibitor to a patient having a Wnt-associated cancer, wherein the DNAPK inhibitor is a compound of formula (I): 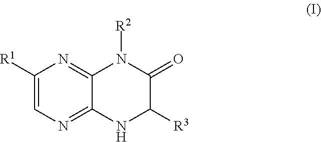
or a pharmaceutically acceptable salt, clathrate, solvate, stereoisomer, tautomer, metabolite, isotopologue or prodrug thereof, wherein:
R¹ is substituted or unsubstituted C₁₋₈ alkyl, substituted or unsubstituted aryl, substituted or unsubstituted cycloalkyl, substituted or unsubstituted heterocyclyl, or substituted or unsubstituted heterocyclylalkyl;
R² is H, substituted or unsubstituted C₁₋₈ alkyl, substituted or unsubstituted cycloalkyl, substituted or unsubstituted heterocyclyl, substituted or unsubstituted heterocyclylalkyl, substituted or unsubstituted aralkyl, or substituted or unsubstituted cycloalkylalkyl;
R³ is H, or a substituted or unsubstituted C₁₋₈ alkyl.

2. The method of claim 1, wherein the Wnt-associated cancer is gastric cancer, breast cancer, endometrial cancer, uterine cancer, colorectal cancer, synovial sarcoma, pancreatic cancer, melanoma, lobular carcinoma, prostate cancer, triple negative breast cancer (TNBC), non-small cell lung cancer (NSCLC), squamous cell lung carcinoma, lung adenocarcinoma, hepatocellular cancer (HCC), ovarian cancer, adenoid carcinoma, adrenocortical carcinoma, bladder/urothelial carcinoma, glioblastoma multiforme (GBM), cervical cancer, head and neck squamous cell carcinoma (HNSCC), kidney cancer, thyroid cancer, acute myelogenous leukemia (AML), acute lymphoblastic leukemia (ALL), multiple myeloma (MM), chronic lymphocytic leukemia (CLL), or chronic myelogenous leukemia.

3. The method of claim 1, wherein said patient is administered about 0.5 mg/day to about 128 mg/day of the DNAPK inhibitor.

4. The method of claim 1, wherein said patient is administered 0.5 mg/day, 1 mg/day, 2 mg/day, 4 mg/day, 8 mg/day, 16 mg/day, 20 mg/day, 30 mg/day, 45 mg/day, 60 mg/day, 90 mg/day, 120 mg/day or 128 mg/day of the DNAPK inhibitor.

5. The method of claim 1, wherein said patient is administered a unit dosage form comprising 0.25 mg, 1.0 mg, 5.0 mg, 7.5 mg, or 10 mg of the DNAPK inhibitor.

6. The method of claim 1, further comprising administering a Wnt pathway modulator, a Wnt inhibitor or an AR antagonist

7. (canceled)

8. The method of claim 1, wherein the DNAPK inhibitor is not

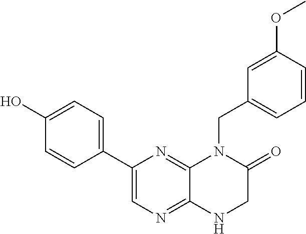 [SMILES: COc1cccc(CN2C(=O)CNc3ncc(-c4ccc(O)cc4)nc32)c1]

9. The method of claim 1, wherein the DNAPK inhibitor is:
7-(5-fluoro-2-methyl-4-(1H-1,2,4-triazol-3-yl)phenyl)-1-((trans-4-methoxycyclohexyl)methyl)-3,4-dihydropyrazino[2,3-b]pyrazin-2(1H)-one;
7-(6-(1H-1,2,4-triazol-3-yl)pyridin-3-yl)-1-(cis-4-methoxycyclohexyl)-3,4-dihydropyrazino[2,3-b]pyrazin-2(1H)-one;
7-(1H-pyrrolo[2,3-b]pyridin-3-yl)-1-(2-(tetrahydro-2H-pyran-4-yl)ethyl)-3,4-dihydropyrazino[2,3-b]pyrazin-2(1H)-one;
7-(5-fluoro-2-methyl-4-(1H-1,2,4-triazol-3-yl)phenyl)-1-((cis-4-methoxycyclohexyl)methyl)-3,4-dihydropyrazino[2,3-b]pyrazin-2(1H)-one;
1-ethyl-7-(1H-pyrrolo[3,2-b]pyridin-5-yl)-3,4-dihydropyrazino[2,3-b]pyrazin-2(1H)-one;
7-(6-(1H-1,2,4-triazol-3-yl)pyridin-3-yl)-1-((cis-4-methoxycyclohexyl)methyl)-3,4-dihydropyrazino[2,3-b]pyrazin-2(1H)-one;
7-(1H-benzo[d]imidazol-4-yl)-1-(2-(tetrahydro-2H-pyran-4-yl)ethyl)-3,4-dihydropyrazino[2,3-b]pyrazin-2(1H)-one;
7-(1H-pyrrolo[2,3-b]pyridin-4-yl)-1-(2-(tetrahydro-2H-pyran-4-yl)ethyl)-3,4-dihydropyrazino[2,3-b]pyrazin-2(1H)-one;
7-(6-(1H-1,2,4-triazol-3-yl)pyridin-3-yl)-1-((trans-4-methoxycyclohexyl)methyl)-3,4-dihydropyrazino[2,3-b]pyrazin-2(1H)-one;
7-(6-(1H-1,2,4-triazol-3-yl)pyridin-3-yl)-1-((trans-4-hydroxycyclohexyl)methyl)-3,4-dihydropyrazino[2,3-b]pyrazin-2(1H)-one;
7-(6-(1H-1,2,4-triazol-3-yl)pyridin-3-yl)-1-(cis-4-hydroxycyclohexyl)-3,4-dihydropyrazino[2,3-b]pyrazin-2(1H)-one;
7-(5-fluoro-2-methyl-4-(1H-1,2,4-triazol-3-yl)phenyl)-1-(cis-4-hydroxycyclohexyl)-3,4-dihydropyrazino[2,3-b]pyrazin-2(1H)-one;
7-(6-(1H-1,2,4-triazol-3-yl)pyridin-3-yl)-1-(tetrahydro-2H-pyran-4-yl)-3,4-dihydropyrazino[2,3-b]pyrazin-2(1H)-one;
7-(6-(1H-1,2,4-triazol-3-yl)pyridin-3-yl)-1-(2-methoxyethyl)-3,4-dihydropyrazino[2,3-b]pyrazin-2(1H)-one;
7-(6-(1H-1,2,4-triazol-3-yl)pyridin-3-yl)-1-ethyl-3,4-dihydropyrazino[2,3-b]pyrazin-2(1H)-one;
7-(5-fluoro-2-methyl-4-(1H-1,2,4-triazol-3-yl)phenyl)-1-((cis-4-hydroxycyclohexyl)methyl)-3,4-dihydropyrazino[2,3-b]pyrazin-2(1H)-one;
7-(5-fluoro-2-methyl-4-(1H-1,2,4-triazol-3-yl)phenyl)-1-(tetrahydro-2H-pyran-4-yl)-3,4-dihydropyrazino[2,3-b]pyrazin-2(1H)-one;
7-(1H-indol-4-yl)-1-(2-(tetrahydro-2H-pyran-4-yl)ethyl)-3,4-dihydropyrazino[2,3-b]pyrazin-2(1H)-one;
7-(5-fluoro-2-methyl-4-(1H-1,2,4-triazol-3-yl)phenyl)-1-((trans-4-hydroxycyclohexyl)methyl)-3,4-dihydropyrazino[2,3-b]pyrazin-2(1H)-one;
7-(6-(1H-1,2,4-triazol-3-yl)pyridin-3-yl)-1-((cis-4-hydroxycyclohexyl)methyl)-3,4-dihydropyrazino[2,3-b]pyrazin-2(1H)-one;
7-(6-(1H-1,2,4-triazol-3-yl)pyridin-3-yl)-1-(trans-4-hydroxycyclohexyl)-3,4-dihydropyrazino[2,3-b]pyrazin-2(1H)-one;
7-(6-(1H-1,2,4-triazol-3-yl)pyridin-3-yl)-1-(trans-4-methoxycyclohexyl)-3,4-dihydropyrazino[2,3-b]pyrazin-2(1H)-one;
7-(6-(1H-1,2,4-triazol-3-yl)pyridin-3-yl)-1-isopropyl-3,4-dihydropyrazino[2,3-b]pyrazin-2(1H)-one;
7-(5-fluoro-2-methyl-4-(1H-1,2,4-triazol-3-yl)phenyl)-1-(trans-4-methoxycyclohexyl)-3,4-dihydropyrazino[2,3-b]pyrazin-2(1H)-one;
7-(5-fluoro-2-methyl-4-(1H-1,2,4-triazol-3-yl)phenyl)-1-(trans-4-hydroxycyclohexyl)-3,4-dihydropyrazino[2,3-b]pyrazin-2(1H)-one;
7-(5-fluoro-2-methyl-4-(1H-1,2,4-triazol-3-yl)phenyl)-1-(2-methoxyethyl)-3,4-dihydropyrazino[2,3-b]pyrazin-2(1H)-one;
7-(5-fluoro-2-methyl-4-(1H-1,2,4-triazol-3-yl)phenyl)-1-isopropyl-3,4-dihydropyrazino[2,3-b]pyrazin-2(1H)-one;
1-ethyl-7-(5-fluoro-2-methyl-4-(1H-1,2,4-triazol-3-yl)phenyl)-3,4-dihydropyrazino[2,3-b]pyrazin-2(1H)-one;
7-(2-hydroxypyridin-4-yl)-1-(2-(tetrahydro-2H-pyran-4-yl)ethyl)-3,4-dihydropyrazino[2,3-b]pyrazin-2(1H)-one;
1-isopropyl-7-(4-methyl-6-(1H-1,2,4-triazol-3-yl)pyridin-3-yl)-3,4-dihydropyrazino[2,3-b]pyrazin-2(1H)-one;
5-(8-isopropyl-7-oxo-5,6,7,8-tetrahydropyrazino[2,3-b]pyrazin-2-yl)-4-methylpicolinamide;
7-(1H-indazol-4-yl)-1-(2-(tetrahydro-2H-pyran-4-yl)ethyl)-3,4-dihydropyrazino[2,3-b]pyrazin-2(1H)-one;
7-(2-aminopyrimidin-5-yl)-1-(2-(tetrahydro-2H-pyran-4-yl)ethyl)-3,4-dihydropyrazino[2,3-b]pyrazin-2(1H)-one;
7-(2-aminopyridin-4-yl)-1-(2-(tetrahydro-2H-pyran-4-yl)ethyl)-3,4-dihydropyrazino[2,3-b]pyrazin-2(1H)-one;
7-(6-(methylamino)pyridin-3-yl)-1-(2-(tetrahydro-2H-pyran-4-yl)ethyl)-3,4-dihydropyrazino[2,3-b]pyrazin-2(1H)-one;
7-(6-hydroxypyridin-3-yl)-1-(2-(tetrahydro-2H-pyran-4-yl)ethyl)-3,4-dihydropyrazino[2,3-b]pyrazin-2(1H)-one;
7-(4-(1H-pyrazol-3-yl)phenyl)-1-(2-methoxyethyl)-3,4-dihydropyrazino[2,3-b]pyrazin-2(1H)-one;
7-(pyridin-3-yl)-1(2-(tetrahydro-2H-pyran-4-yl)ethyl)-3,4-dihydropyrazino[2,3-b]pyrazin-2(1H)-one;
7-(1H-indazol-4-yl)-1-(2-methoxyethyl)-3,4-dihydropyrazino[2,3-b]pyrazin-2(1H)-one;
7-(1H-indazol-6-yl)-1-(2-methoxyethyl)-3,4-dihydropyrazino[2,3-b]pyrazin-2(1H)-one;
7-(pyrimidin-5-yl)-1-(2-(tetrahydro-2H-pyran-4-yl)ethyl)-3,4-dihydropyrazino[2,3-b]pyrazin-2(1H)-one;
7-(6-methoxypyridin-3-yl)-1-(2-(tetrahydro-2H-pyran-4-yl)ethyl)-3,4-dihydropyrazino[2,3-b]pyrazin-2(1H)-one;
1-(2-methoxyethyl)-7-(1H-pyrrolo[2,3-b]pyridin-5-yl)-3,4-dihydropyrazino[2,3-b]pyrazin-2(1H)-one;
1-ethyl-7-(1H-pyrrolo[2,3-b]pyridin-5-yl)-3,4-dihydropyrazino[2,3-b]pyrazin-2(1H)-one;
1-ethyl-7-(1H-indazol-4-yl)-3,4-dihydropyrazino[2,3-b]pyrazin-2(1H)-one;
7-(pyridin-4-yl)-1-(2-(tetrahydro-2H-pyran-4-yl)ethyl)-3,4-dihydropyrazino[2,3-b]pyrazin-2(1H)-one;
7-(6-aminopyridin-3-yl)-1-(2-(tetrahydro-2H-pyran-4-yl)ethyl)-3,4-dihydropyrazino[2,3-b]pyrazin-2(1H)-one;
1-methyl-7-(2-methyl-6-(4H-1,2,4-triazol-3-yl)pyridin-3-yl)-3,4-dihydropyrazino[2,3-b]pyrazin-2(1H)-one;
2-(2-hydroxypropan-2-yl)-5-(8-(trans-4-methoxycyclohexyl)-7-oxo-5,6,7,8-tetrahydropyrazino[2,3-b]pyrazin-2-yl)pyridine 1-oxide;
4-methyl-5-(7-oxo-8-((tetrahydro-2H-pyran-4-yl)methyl)-5,6,7,8-tetrahydropyrazino[2,3-b]pyrazin-2-yl)picolinamide;
5-(8-((cis-4-methoxycyclohexyl)methyl)-7-oxo-5,6,7,8-tetrahydropyrazino[2,3-b]pyrazin-2-yl)-4-methylpicolinamide;
7-(1H-pyrazol-4-yl)-1-(2-(tetrahydro-2H-pyran-4-yl)ethyl)-3,4-dihydropyrazino[2,3-b]pyrazin-2(1H)-one;
1-(trans-4-methoxycyclohexyl)-7-(4-methyl-6-(1H-1,2,4-triazol-3-yl)pyridin-3-yl)-3,4-dihydropyrazino[2,3-b]pyrazin-2(1H)-one;
3-((7-(2-methyl-6-(4H-1,2,4-triazol-3-yl)pyridin-3-yl)-2-oxo-3,4-dihydropyrazino[2,3-b]pyrazin-1(2H)-yl)methyl)benzonitrile;
1-((trans-4-methoxycyclohexyl)methyl)-7-(4-methyl-6-(1H-1,2,4-triazol-3-yl)pyridin-3-yl)-3,4-dihydropyrazino[2,3-b]pyrazin-2(1H)-one;
3-(7-oxo-8-(2-(tetrahydro-2H-pyran-4-yl)ethyl)-5,6,7,8-tetrahydropyrazino[2,3-b]pyrazin-2-yl)benzamide;
5-(8-((trans-4-methoxycyclohexyl)methyl)-7-oxo-5,6,7,8-tetrahydropyrazino[2,3-b]pyrazin-2-yl)-4-methylpicolinamide;
3-((7-(6-(2-hydroxypropan-2-yl)pyridin-3-yl)-2-oxo-3,4-dihydropyrazino[2,3-b]pyrazin-1(2H)-yl)methyl)benzonitrile;
7-(6-(2-hydroxypropan-2-yl)pyridin-3-yl)-1-((1R,3R)-3-methoxycyclopentyl)-3,4-dihydropyrazino[2,3-b]pyrazin-2(1H)-one;
7-(6-(2-hydroxypropan-2-yl)pyridin-3-yl)-1-((1 S,3R)-3-methoxycyclopentyl)-3,4-dihydropyrazino[2,3-b]pyrazin-2(1H)-one;
7-(6-(2-hydroxypropan-2-yl)pyridin-3-yl)-1-((1 S,3 S)-3-methoxycyclopentyl)-3,4-dihydropyrazino[2,3-b]pyrazin-2(1H)-one;
7-(6-(2-hydroxypropan-2-yl)pyridin-3-yl)-1-((1R,3 S)-3-methoxycyclopentyl)-3,4-dihydropyrazino[2,3-b]pyrazin-2(1H)-one;
7-(1H-indazol-6-yl)-1-(2-(tetrahydro-2H-pyran-4-yl)ethyl)-3,4-dihydropyrazino[2,3-b]pyrazin-2(1H)-one;
7-(2-methyl-6-(4H-1,2,4-triazol-3-yl)pyridin-3-yl)-1-(2-morpholinoethyl)-3,4-dihydropyrazino[2,3-b]pyrazin-2(1H)-one;
1-(trans-4-hydroxycyclohexyl)-7-(2-methyl-6-(4H-1,2,4-triazol-3-yl)pyridin-3-yl)-3,4-dihydropyrazino[2,3-b]pyrazin-2(1H)-one;
1-(cis-4-hydroxycyclohexyl)-7-(2-methyl-6-(4H-1,2,4-triazol-3-yl)pyridin-3-yl)-3,4-dihydropyrazino[2,3-b]pyrazin-2(1H)-one;
7-(6-(2-hydroxypropan-2-yl)pyridin-3-yl)-1-(2-morpholinoethyl)-3,4-dihydropyrazino[2,3-b]pyrazin-2(1H)-one;
1-isopropyl-7-(2-methyl-6-(4H-1,2,4-triazol-3-yl)pyridin-3-yl)-3,4-dihydropyrazino[2,3-b]pyrazin-2(1H)-one;
7-(1H-imidazo[4,5-b]pyridin-6-yl)-1-(2-(tetrahydro-2H-pyran-4-yl)ethyl)-3,4-dihydropyrazino[2,3-b]pyrazin-2(1H)-one;
1-((cis-4-methoxycyclohexyl)methyl)-7-(2-methyl-6-(1H-1,2,4-triazol-3-yl)pyridin-3-yl)-3,4-dihydropyrazino[2,3-b]pyrazin-2(1H)-one;
1-(trans-4-hydroxycyclohexyl)-7-(6-(2-hydroxypropan-2-yl)pyridin-3-yl)-3,4-dihydropyrazino[2,3-b]pyrazin-2(1H)-one;
1-(cis-4-hydroxycyclohexyl)-7-(6-(2-hydroxypropan-2-yl)pyridin-3-yl)-3,4-dihydropyrazino[2,3-b]pyrazin-2(1H)-one;
4-(7-oxo-8-(2-(tetrahydro-2H-pyran-4-yl)ethyl)-5,6,7,8-tetrahydropyrazino[2,3-b]pyrazin-2-yl)benzamide;
7-(1H-indazol-5-yl)-1-(2-(tetrahydro-2H-pyran-4-yl)ethyl)-3,4-dihydropyrazino[2,3-b]pyrazin-2(1H)-one;
7-(1H-pyrrolo[2,3-b]pyridin-5-yl)-1-(2-(tetrahydro-2H-pyran-4-yl)ethyl)-3,4-dihydropyrazino[2,3-b]pyrazin-2(1H)-one;
7-(2-methyl-6-(4H-1,2,4-triazol-3-yl)pyridin-3-yl)-1-(tetrahydro-2H-pyran-4-yl)-3,4-dihydropyrazino[2,3-b]pyrazin-2(1H)-one;
1-((1 S,3R)-3-methoxycyclopentyl)-7-(2-methyl-6-(4H-1,2,4-triazol-3-yl)pyridin-3-yl)-3,4-dihydropyrazino[2,3-b]pyrazin-2(1H)-one;
1-((1R,3R)-3-methoxycyclopentyl)-7-(2-methyl-6-(4H-1,2,4-triazol-3-yl)pyridin-3-yl)-3,4-dihydropyrazino[2,3-b]pyrazin-2(1H)-one;
1-((1R,3 S)-3-methoxycyclopentyl)-7-(2-methyl-6-(4H-1,2,4-triazol-3-yl)pyridin-3-yl)-3,4-dihydropyrazino[2,3-b]pyrazin-2(1H)-one;
1-((1 S,3 S)-3-methoxycyclopentyl)-7-(2-methyl-6-(4H-1,2,4-triazol-3-yl)pyridin-3-yl)-3,4-dihydropyrazino[2,3-b]pyrazin-2(1H)-one;
7-(1H-indol-5-yl)-1-(2-(tetrahydro-2H-pyran-4-yl)ethyl)-3,4-dihydropyrazino[2,3-b]pyrazin-2(1H)-one;
1-ethyl-7-(2-methyl-6-(4H-1,2,4-triazol-3-yl)pyridin-3-yl)-3,4-dihydropyrazino[2,3-b]pyrazin-2(1H)-one;
7-(1H-indol-6-yl)-1-(2-(tetrahydro-2H-pyran-4-yl)ethyl)-3,4-dihydropyrazino[2,3-b]pyrazin-2(1H)-one;
7-(4-(2-hydroxypropan-2-yl)phenyl)-1-(trans-4-methoxycyclohexyl)-3,4-dihydropyrazino[2,3-b]pyrazin-2(1H)-one;
7-(6-(2-hydroxypropan-2-yl)pyridin-3-yl)-1-(tetrahydro-2H-pyran-4-yl)-3,4-dihydropyrazino[2,3-b]pyrazin-2(1H)-one;
1-((trans-4-methoxycyclohexyl)methyl)-7-(2-methyl-6-(1H-1,2,4-triazol-3-yl)pyridin-3-yl)-3,4-dihydropyrazino[2,3-b]pyrazin-2(1H)-one;
7-(6-(2-hydroxypropan-2-yl)pyridin-3-yl)-1-((cis-4-methoxycyclohexyl)methyl)-3,4-dihydropyrazino[2,3-b]pyrazin-2(1H)-one;
1-(2-methoxyethyl)-7-(4-methyl-2-(methylamino)-1H-benzo[d]imidazol-6-yl)-3,4-dihydropyrazino[2,3-b]pyrazin-2(1H)-one;
7-(7-methyl-2-oxo-2,3-dihydro-1H-benzo[d]imidazol-5-yl)-1-((tetrahydro-2H-pyran-4-yl)methyl)-3,4-dihydropyrazino[2,3-b]pyrazin-2(1H)-one;
7-(2-methyl-4-(4H-1,2,4-triazol-3-yl)phenyl)-3,4-dihydropyrazino[2,3-b]pyrazin-2(1H)-one;
1-(2-methoxyethyl)-7-(4-methyl-6-(1H-1,2,4-triazol-3-yl)pyridin-3-yl)-3,4-dihydropyrazino[2,3-b]pyrazin-2(1H)-one;
1-benzyl-7-(2-methyl-4-(4H-1,2,4-triazol-3-yl)phenyl)-3,4-dihydropyrazino[2,3-b]pyrazin-2(1H)-one;
7-(3-fluoro-4-(4H-1,2,4-triazol-3-yl)phenyl)-1-(2-methoxyethyl)-3,4-dihydropyrazino[2,3-b]pyrazin-2(1H)-one;
7-(3-fluoro-4-(4H-1,2,4-triazol-3-yl)phenyl)-1-(2-(tetrahydro-2H-pyran-4-yl)ethyl)-3,4-dihydropyrazino[2,3-b]pyrazin-2(1H)-one;
7-(3-fluoro-2-methyl-4-(1H-1,2,4-triazol-3-yl)phenyl)-1-(2-methoxyethyl)-3,4-dihydropyrazino[2,3-b]pyrazin-2(1H)-one;
1-(trans-4-methoxycyclohexyl)-7-(2-methyl-6-(4H-1,2,4-triazol-3-yl)pyridin-3-yl)-3,4-dihydropyrazino[2,3-b]pyrazin-2(1H)-one;
7-(6-(2-hydroxypropan-2-yl)pyridin-3-yl)-1-(trans-4-methoxycyclohexyl)-3,4-dihydropyrazino[2,3-b]pyrazin-2(1H)-one;
7-(5-fluoro-2-methyl-4-(4H-1,2,4-triazol-3-yl)phenyl)-1-(2-(tetrahydro-2H-pyran-4-yl)ethyl)-3,4-dihydropyrazino[2,3-b]pyrazin-2(1H)-one;
7-(3-fluoro-2-methyl-4-(1H-1,2,4-triazol-3-yl)phenyl)-1-(2-(tetrahydro-2H-pyran-4-yl)ethyl)-3,4-dihydropyrazino[2,3-b]pyrazin-2(1H)-one;
1-(2-methoxyethyl)-7-(2-methyl-6-(4H-1,2,4-triazol-3-yl)pyridin-3-yl)-3,4-dihydropyrazino[2,3-b]pyrazin-2(1H)-one;
7-(6-(2-hydroxypropan-2-yl)pyridin-3-yl)-1-((trans-4-methoxycyclohexyl)methyl)-3,4-dihydropyrazino[2,3-b]pyrazin-2(1H)-one;
1-(cyclopentylmethyl)-7-(6-(2-hydroxypropan-2-yl)pyridin-3-yl)-3,4-dihydropyrazino[2,3-b]pyrazin-2(1H)-one;
7-(4-(2-hydroxypropan-2-yl)phenyl)-1-(2-methoxyethyl)-3,4-dihydropyrazino[2,3-b]pyrazin-2(1H)-one;
(S)-7-(6-(1-hydroxyethyl)pyridin-3-yl)-1-(2-(tetrahydro-2H-pyran-4-yl)ethyl)-3,4-dihydropyrazino[2,3-b]pyrazin-2(1H)-one;
(R)-7-(6-(1-hydroxyethyl)pyridin-3-yl)-1-(2-(tetrahydro-2H-pyran-4-yl)ethyl)-3,4-dihydropyrazino[2,3-b]pyrazin-2(1H)-one;
7-(2-methyl-6-(4H-1,2,4-triazol-3-yl)pyridin-3-yl)-1-((tetrahydro-2H-pyran-4-yl)methyl)-3,4-dihydropyrazino[2,3-b]pyrazin-2(1H)-one;
7-(4-(2-hydroxypropan-2-yl)phenyl)-1-(2-(tetrahydro-2H-pyran-4-yl)ethyl)-3,4-dihydropyrazino[2,3-b]pyrazin-2(1H)-one;
7-(6-(2-hydroxypropan-2-yl)pyridin-3-yl)-1-(4-(trifluoromethyl)benzyl)-3,4-dihydropyrazino[2,3-b]pyrazin-2(1H)-one;
7-(6-(2-hydroxypropan-2-yl)pyridin-3-yl)-1-(3-(trifluoromethyl)benzyl)-3,4-dihydropyrazino[2,3-b]pyrazin-2(1H)-one;
7-(6-(2-hydroxypropan-2-yl)pyridin-3-yl)-1-(3-methoxypropyl)-3,4-dihydropyrazino[2,3-b]pyrazin-2(1H)-one;
7-(4-methyl-6-(1H-1,2,4-triazol-3-yl)pyridin-3-yl)-1-(2-(tetrahydro-2H-pyran-4-yl)ethyl)-3,4-dihydropyrazino[2,3-b]pyrazin-2(1H)-one;
7-(6-(2-hydroxypropan-2-yl)pyridin-3-yl)-1-(2-methoxyethyl)-3,4-dihydropyrazino[2,3-b]pyrazin-2(1H)-one;
7-(6-(2-hydroxypropan-2-yl)pyridin-3-yl)-1-((tetrahydro-2H-pyran-4-yl)methyl)-3,4-dihydropyrazino[2,3-b]pyrazin-2(1H)-one;
7-(4-methyl-2-(methylamino)-1H-benzo[d]imidazol-6-yl)-1-((tetrahydro-2H-pyran-4-yl)methyl)-3,4-dihydropyrazino[2,3-b]pyrazin-2(1H)-one;
7-(2-amino-4-methyl-1H-benzo[d]imidazol-6-yl)-1-((tetrahydro-2H-pyran-4-yl)methyl)-3,4-dihydropyrazino[2,3-b]pyrazin-2(1H)-one;
7-(2-methyl-6-(4H-1,2,4-triazol-3-yl)pyridin-3-yl)-1-(2-(tetrahydro-2H-pyran-4-yl)ethyl)-3,4-dihydropyrazino[2,3-b]pyrazin-2(1H)-one;
(R)-7-(6-(2-hydroxypropan-2-yl)pyridin-3-yl)-3-methyl-1-(2-(tetrahydro-2H-pyran-4-yl)ethyl)-3,4-dihydropyrazino[2,3-b]pyrazin-2(1H)-one;
(S)-7-(6-(2-hydroxypropan-2-yl)pyridin-3-yl)-3-methyl-1-(2-(tetrahydro-2H-pyran-4-yl)ethyl)-3,4-dihydropyrazino[2,3-b]pyrazin-2(1H)-one;
7-(6-(2-hydroxypropan-2-yl)pyridin-3-yl)-3,3-dimethyl-1-(2-(tetrahydro-2H-pyran-4-yl)ethyl)-3,4-dihydropyrazino[2,3-b]pyrazin-2(1H)-one;
7-(2-amino-4-methyl-1H-benzo[d]imidazol-6-yl)-1-(2-(tetrahydro-2H-pyran-4-yl)ethyl)-3,4-dihydropyrazino[2,3-b]pyrazin-2(1H)-one;
7-(6-(2-hydroxypropan-2-yl)pyridin-3-yl)-1-(2-(tetrahydro-2H-pyran-4-yl)ethyl)-3,4-dihydropyrazino[2,3-b]pyrazin-2(1H)-one;
7-(2-methyl-4-(1H-1,2,4-triazol-3-yl)phenyl)-1-(2-(tetrahydro-2H-pyran-4-yl)ethyl)-3,4-dihydropyrazino[2,3-b]pyrazin-2(1H)-one;
7-(4-(1H-1,2,4-triazol-5-yl)phenyl)-1-(2-(tetrahydro-2H-pyran-4-yl)ethyl)-3,4-dihydropyrazino[2,3-b]pyrazin-2(1H)-one;
1-(1-hydroxypropan-2-yl)-7-(2-methyl-6-(1H-1,2,4-triazol-3-yl)pyridin-3-yl)-3,4-dihydropyrazino[2,3-b]pyrazin-2(1H)-one; or
1-(2-hydroxyethyl)-7-(2-methyl-6-(1H-1,2,4-triazol-3-yl)pyridin-3-yl)-3,4-dihydropyrazino[2,3-b]pyrazin-2(1H)-one,
or a pharmaceutically acceptable salt, clathrate, solvate, stereoisomer, tautomer, metabolite, isotopologue or prodrug thereof.

10. The method of claim 1, wherein the DNAPK inhibitor is 1-ethyl-7-(2-methyl-6-(1H-1,2,4-triazol-3-yl)pyridin-3-yl)-3,4-dihydropyrazino[2,3-b]pyrazin-2(1H)-one, or a tautomer thereof, for example, 1-ethyl-7-(2-methyl-6-(4H-1,2,4-triazol-3-yl)pyridin-3-yl)-3,4-dihydropyrazino[2,3-b]pyrazin-2(1H)-one, or 1-ethyl-7-(2-methyl-6-(1H-1,2,4-triazol-5-yl)pyridin-3-yl)-3,4-dihydropyrazino[2,3-b]pyrazin-2(1H)-one.
